(19) United States
(12) Patent Application Publication
(10) Pub. No.: US 2020/0388385 A1
(43) Pub. Date: Dec. 10, 2020

(54) EFFICIENT DIAGNOSIS CONFIRMATION OF A SUSPECT CONDITION FOR CERTIFICATION AND/OR RE-CERTIFICATION BY A CLINICIAN

(71) Applicant: EMBLEMHEALTH, INC., New York, NY (US)

(72) Inventors: JEFFREY J. DE LOS REYES, Scarsdale, NY (US); THOMAS A. MACMILLAN, Summit, NJ (US); JULIE F. SMITH, Tuckahoe, NY (US)

(21) Appl. No.: 16/434,183

(22) Filed: Jun. 7, 2019

(51) Int. Cl. G16H 50/20; G16H 10/60; G16H 30/20

(52) U.S. Cl. CPC G16H 50/20; G16H 30/20; G16H 10/60

(57) ABSTRACT

Disclosed is a method, a device, a system and/or a manufacture of efficient confirmation of a suspect condition for certification and/or re-certification by a clinician. In one embodiment, a method includes extracting a health data of the patient from an electronic medical record (EMR) and applying an assessment ruleset and/or a re-certification evaluation to generate a suspect health condition. An EMR request for health information generated within a native encounter between the patient and the clinician is generated by a computing device of the clinician. The suspect condition data is then integrated in visual association with diagnosed condition(s) within the clinical documentation workflow of an application running on the computing device. A condition certification is received from the computing device and a new record created in the EMR entered and/or a re-certification specified. An updated health data may be assessed in real time and/or automatically detect compliance with documentation requirements.

### FIELD OF TECHNOLOGY

This disclosure relates generally to data processing devices and, more particularly, to a method, a device, and/or a system of efficient confirmation of a suspect condition for certification and/or re-certification by a clinician.

### BACKGROUND

Diagnosing health conditions may be one of the most important functions of a healthcare provider. Accuracy and efficiency in the diagnosis process are important not only for the benefit of the healthcare provider as an organization, but for the healthcare professionals working with the healthcare provider and for the patients the healthcare provider serves. Accuracy promotes patient wellness and reduces risk of liability for a missed diagnosis. A healthcare provider can increase revenue and receive faster insurance reimbursement by ensuring conditions are accurately and efficiently identified, treated, and documented. Efficiency ensures healthcare professionals require less time to certify a diagnosis, which leaves more time for patients and/or permits more patients to be seen by healthcare professionals. This in turn can increase the capacity and revenue of the healthcare provider.

Two important information technologies that may support the diagnosis process are an electronic medical record system and a point-of-care (POC) software application. An electronic medical record (EMR) maintains and organizes all relevant health information of the patient, for example confirmed diagnoses, test results, prescription medication tracking, notes, and other health-related data. The POC application presents EMR information to a healthcare professional in association with a patient encounter such as an appointment. The POC application may include clinical documentation workflows that assist the clinician in reviewing the EMR and diagnosing and/or certifying a health condition. Many diagnoses in the EMR may originate as a suspect condition that is evaluated with the assistance of the POC application.

In what may be a familiar example, a patient may experience a symptom and make an appointment with a clinician. The clinician may use his or her experience to evaluate the symptom and then determine it arises from a health condition that the clinician suspects the patient may have, which may be referred to as a suspect condition. The clinician then diagnoses the condition with his or her professional training, possibly through additional acts that may help to certify the diagnosis (e.g., a clinical laboratory test).

Challenges may be evident for healthcare professionals in a clinical environment who may experience significant time constraints in reviewing an EMR of a patient, evaluating a symptom, diagnosing a condition, and/or treating the condition. The healthcare professional may see multiple patients with the same or similar conditions each day and could possibly confuse patients and their medical histories. On the other hand, a healthcare professional may see a patient with a condition they do not regularly diagnose, which may slow the diagnosis process and decrease accuracy. The EMR may be large and include many different records that are difficult to assess when evaluating a suspect condition. The healthcare professional may not properly certify a diagnosis, for example, because they are unaware of a change in approved diagnosis procedure. The healthcare professional may fail to properly certify a diagnosis because they may be unaware of new documentation requirements (e.g., a contractual requirement of an insurance company or a mandated government regulation). In some cases, the healthcare professional may just forget to re-certify an ongoing condition, even when necessary to insurance reimbursement or when such certification is necessary for insurance company risk-adjustment purposes. In addition, significant responsibility may attach to a healthcare provider once a suspect condition is identified, even if generated automatically.

At the organizational level, these challenges may manifest as poor patient outcomes (some of which may be life threatening), reduced health professional efficiency, increased risk of malpractice liability, missed insurance reimbursement claims, relationship management challenges with insurance companies, and overall declined revenue. Therefore, there is a continuing need for technologies that increase the accuracy and efficiency of the diagnosis process.

### SUMMARY

Disclosed are a method, a device, and/or a system of efficient confirmation of a suspect condition for certification and/or re-certification by a clinician. The present embodiments may automatically generate a suspect condition and then place the suspect condition in context within a time, a physical space (e.g., a native encounter that is in person), and/or a visual space (e.g., on a user interface relative to what may be key information in the EMR). Resources for completing the diagnosis accurately and efficiently can be automatically provided to the clinician, including providing options for diagnosis, referral, testing, and collecting additional information. In one or more embodiments, suspect conditions can be determined from new information in real time, and documentation of the EMR can be automatically evaluated based on documentation requirements, for example to determine whether records are in compliance with internal quality controls or government regulation.

In one embodiment, a method for accurately and efficiently confirming a suspected health condition of a patient includes extracting health data of the patient from an electronic medical record of the patient. The health data includes a diagnosed condition, a quantitative test result, and/or a qualitative test result. An assessment ruleset is applied to the health data of the patient to output a suspect condition based on the health data. The suspect condition is a potential health condition diagnosable by a clinician. A suspect condition data is then generated that includes a name of the suspect condition, a description of the suspect condition, and/or a condition ID such as a hierarchical condition category code.

The method detects an EMR request generated by a POC application running on a computing device of a clinician requesting information of the electronic medical record of the patient. The POC application includes a clinical documentation workflow. The information of the electronic medical record includes one or more diagnosed conditions and is presented to the clinician through a user interface of the POC application within the clinical documentation workflow. The information of the electronic medical record is transmitted from a server to the computing device over a network, along with the suspect condition data.

The suspect condition data is then integrated in visual association with the diagnosed condition(s) extracted from the electronic medical record within the clinical documentation workflow. The integration provides a context for the suspect condition relative to the diagnosed condition(s) within a native encounter of the clinician and the patient. After the method may present one or more diagnosis options, the method then receives a diagnosis confirmation of the suspect condition from the computing device of the clinician to certify the suspect condition. The suspect condition is converted into the diagnosed condition based on the context for the suspect condition relative to the diagnosed condition(s) within the native encounter by generating a new record in the electronic medical record of the patient.

The method may generate a suspect reasoning data specifying one or more reasons the suspect condition was output from application of the assessment ruleset to the health data of the patient and may also integrate the suspect reasoning data in visual association with the one or more diagnosed conditions. The suspect condition may also be determined to be a care gap of the patient through comparison to the health data or diagnosed conditions in the electronic medical record.

The method may generate a set of certification options in association with the suspect condition data on the user interface. The set of certification options may comprise at least a diagnosis confirmation option, but also may include (i) an absence confirmation option initiating generation of an absence record certifying that the suspect condition is absent from the patient, (ii) a referral order option initiating a referral process to refer the patient to a referral clinician or other healthcare provider qualified to diagnose the suspect condition, (iii) a test order option to initiate ordering a diagnostic test approved to diagnose the suspect condition, and/or (iv) an information collection option to initiate a data collection process gathering additional information usable to diagnose the suspect condition within the native encounter of the clinician and the patient.

The referral order option may initiate referencing a clinician database to determine the referral clinician qualified to diagnose the suspect condition. The test order option may initiate referencing a test database to determine the diagnostic test approved to diagnose the suspect condition. Similarly, a diagnostic procedure database may be referenced to extract a data collection process usable to diagnose the suspect condition. The data collection process may gather additional information usable to diagnose the suspect condition and/or the diagnostic test approved to diagnose the suspect condition.

The method may also collect an updated health data from the patient that includes data generated from the additional information usable to diagnose the suspect condition and/or a test result of the diagnostic test. The assessment ruleset may then be re-applied to the updated health data of the patient to output a second suspect condition based on the updated health data. A second suspect condition data may then be integrated in visual association with the suspect condition within the clinical documentation workflow to provide the context for the suspect condition relative to the second suspect condition within the native encounter of the clinician and the patient.

The method may determine a documentation requirement of the suspect condition and/or the diagnosed condition. The electronic medical record may be queried for a document matching the documentation requirement of the diagnosed condition. A documentation deficiency may be determined in the diagnosed condition based on a failure to return the document matching the documentation requirement. A deficiency notification may then be generated, and optionally integrated in visual association with the diagnosed condition within the native encounter, to direct the clinician to obtain the document meeting the documentation requirement before the native encounter of the clinician and the patient ends.

The method may generate an audit log that the patient is in the presence of the clinician, for example to document the native encounter. The method may store the suspect condition data in a partition of the electronic medical record distinct from the one or more diagnosed conditions, for example to ensure provenance of the EMR. Data of a health tracking device of the patient may be received to generate the updated health data of the patient.

The suspect condition data may include a condition ID that may be a hierarchical condition code and/or a diagnosis code. A patient UID that may be utilized by the method may be a medical record number, a clinical record number, and/or a record set ID. The quantitative test result may include data from a physical characteristic of the patient, a vital sign, and/or a laboratory test. The quantitative test result may be received in a communication protocol that is either FHIR or HL7.

The qualitative test result comprises data from family history evaluation, a patient generated report, a physical exam, an observational narrative, a system review, a verbal screening, a written screening, a psychiatric evaluation, a medical imaging data, and/or a medical graph data. The documentation requirement may be based on a test result, a clinician narrative, an identification of multiple related health conditions, a certification date, a clinician authentication, and/or a care plan. The documentation requirement may be based on data specifying at least one of an internal quality control, a new diagnosis procedure, an insurance claim requirement, and a government regulation.

In another embodiment, a method extracts a health data of the patient from an electronic medical record of the patient, the health data including a diagnosed condition. Occurrence of a re-certification trigger is then determined, the re-certification trigger including a certification date of the diagnosed condition exceeding a threshold time to define a suspect condition. A diagnosis confirmation of the suspect condition may be received from the computing device of the clinician to re-certify the diagnosed condition. The method may then conserve the diagnosed condition by maintaining an existing instance of a condition record of the diagnosed condition in the electronic medical record of the patient while adding a new certification date.

In yet another embodiment, a system includes an record server, a certification server, and a communication network. The record server includes an EMR database storing an electronic medical record and an EMR management application for extracting information of the electronic medical record in response to a query and generating and/or modifying the condition record in response to a record creation instruction.

The certification server includes a suspect condition assessment engine that includes computer readable instructions that when executed on the processor of the certification server: (i) request a health data of the patient from the electronic medical record of the patient, (ii) apply an assessment ruleset to the health data of the patient that outputs a suspect condition based on the health data; and (iii) generate a suspect condition data. The certification server includes a request agent comprising computer readable instructions that when executed on the processor of the certification server detects an EMR request generated by a documentation application running on a computing device of a clinician requesting information of the electronic medical record of the patient. The system further includes a clinician notification routine comprising computer readable instructions that when executed on the processor of the certification server receives a call from the request agent and transmits the suspect condition data in coordination with information of the electronic medical record to the computing device over the network. The system may also include a computing device and a documentation server.

### BRIEF DESCRIPTION OF THE DRAWINGS

The embodiments of this disclosure are illustrated by way of example and not limitation in the figures of the accompanying drawings, in which like references indicate similar elements and in which:

FIG. 1 illustrates a certification network for providing accurate and/or efficient certification of a suspect condition, the network comprising an record server storing an electronic medical record (EMR) of a patient, a certification server for generating a suspect condition data and processing data sufficient to certify the suspect condition data as a diagnosed condition, a computing device running a POC application integrating the suspect condition data with existing EMR data to aid a clinician in diagnosis, and a documentation server for assessing documentation of the diagnosed condition, according to one or more embodiments.

FIG. 2 illustrates the record server of FIG. 1, including an EMR management application, an audit log routine for logging a native encounter between the patient and clinician, and the EMR of the patient comprising a condition record of a diagnosed condition and optionally a partition in which the suspect condition data may be stored, according to one or more embodiments.

FIG. 3 illustrates the certification server of FIG. 1, including a suspect condition assessment engine that applies an assessment ruleset to health data received from the record server of FIG. 2 to generate a suspect condition, a database for storing the suspect condition, and a certification routine for certifying the diagnosis of the suspect condition to generate a record in the EMR, according to one or more embodiments.

FIG. 4 illustrates the computing device of FIG. 1 running the POC application aiding the clinician in diagnosing the patient, the computing device receiving health information from the EMR of FIG. 2 and the suspect condition data generated by the certification server of FIG. 3 and integrating the health information and the suspect condition data, along with diagnosis menu options, in a clinical documentation workflow to assist in certifying the suspect condition, according to one or more embodiments.

FIG. 5 illustrates the documentation server of FIG. 1 for assessing documentation of the suspect condition, the documentation server comprising a documentation requirement database associating a health condition with one or more documentation requirements, a documentation assessment engine for evaluating documentation supporting the suspect condition as a diagnosed condition (and potentially generating a deficiency notification), and a claim generation routine for automatically generating an insurance claim for the diagnosed condition, according to one or more embodiments.

FIG. 6 is a certification process flow illustrating an overview of an accurate and efficient process for efficient confirmation of a suspect condition for certification and/or re-certification by a healthcare professional, according to one or more embodiments.

FIG. 7 is a re-certification process flow illustrating generation of a suspect condition data upon occurrence of a trigger condition, for example expiration of a certification date or change of a diagnosis requirement, according to one or more embodiments.

FIG. 8 is an assessment process flow for determining a suspect condition from application of the assessment ruleset to the health data extracted from the EMR, according to one or more embodiments.

FIG. 9 is a suspect condition process flow illustrating determination of a relevant encounter for presentation of the suspect condition data and transmission of the suspect condition data, for example to the computing device of FIG. 4, according to one or more embodiments.

FIG. 10 is a certification process flow illustrating generation of certification options for integration along with the suspect condition data in the POC application of FIG. 4, including a certification option, a diagnosis confirmation option, an absence confirmation option, a test order option, and a referral order option, according to one or more embodiments.

FIG. 11 is a record storage process flow illustrating a process by which a suspect condition that was converted to a diagnosed condition may be stored as a new record and/or a certification date of an existing record in the EMR database updated, according to one or more embodiments.

FIG. 12 is a documentation assessment process flow illustrating an automatic evaluation of documentation of the diagnosed condition, for example to comply with insurance documentation requirements, according to one or more embodiments.

FIG. 13 illustrates a first part of an example embodiment in which a clinician re-certifies a diabetes diagnosis and certifies diabetes-related complications, according to one or more embodiments.

FIG. 14 illustrates a second part of the example of FIG. 13, according to one or more embodiments.

Other features of the present embodiments will be apparent from the accompanying drawings and from the detailed description that follows.

### DETAILED DESCRIPTION

Disclosed are a method, a device, and system of efficient confirmation of a suspect condition for certification and/or re-certification by a clinician. Although the present embodiments have been described with reference to specific example embodiments, it will be evident that various modifications and changes may be made to these embodiments without departing from the broader spirit and scope of the various embodiments.

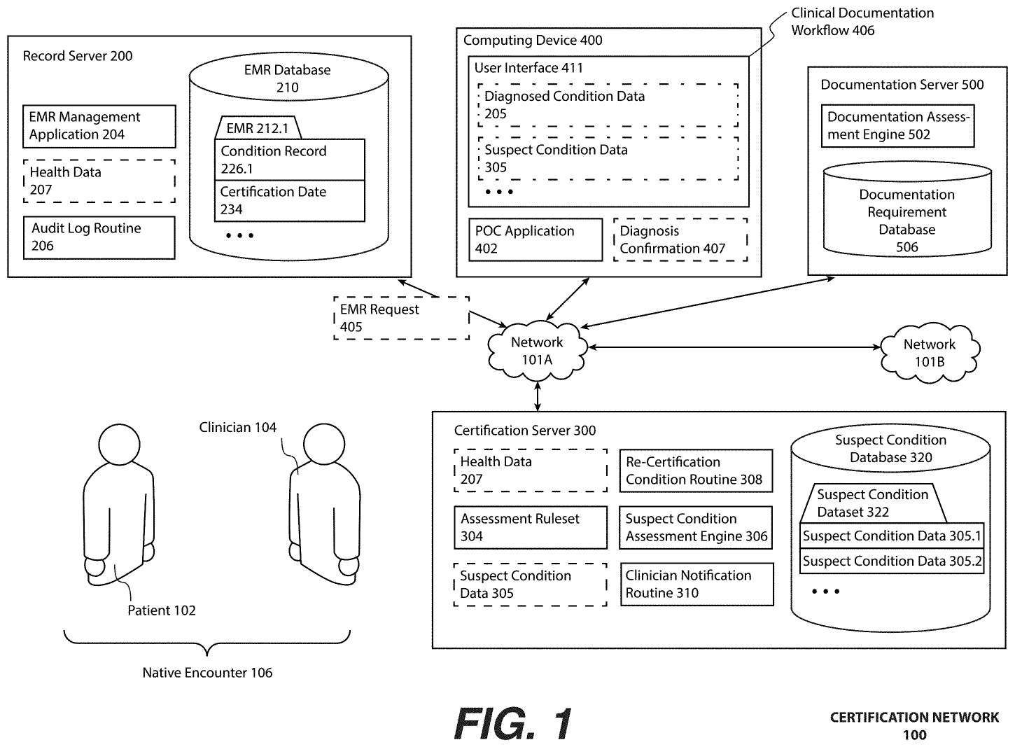

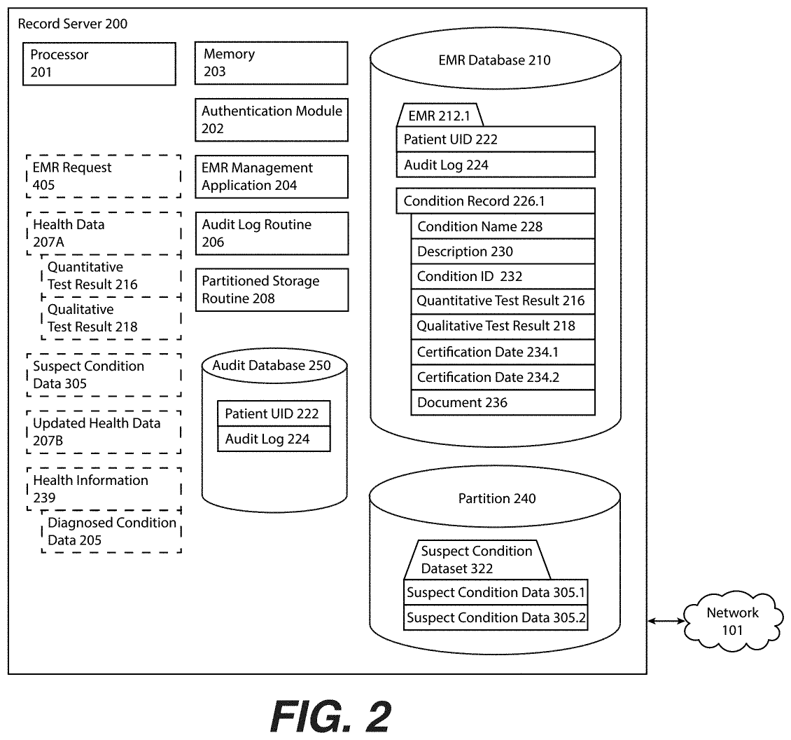

FIG. 1 illustrates a certification network 100 for providing accurate and/or efficient certification of a suspect condition. The certification network 100 is a system of two or more elements communicating through a communication network (i.e., the network 101) such as a virtual private network (VPN) utilized by a healthcare provider. The healthcare provider may be, for example, a medical practice, a hospital, an outpatient facility, a health system, a health maintenance organization (HMO), and/or an accountable care organization (ACO) An record server 200 may store an electronic medical record 212 (i.e., the EMR 212) of a patient 102. A certification server 300 may generate a suspect condition represented by a suspect condition data 305. The certification server 300 may process a diagnosis confirmation 407 of a clinician 104 sufficient to certify the suspect condition as a diagnosed condition represented by a diagnosed condition data 205. A computing device 400 (e.g., a tablet) comprises a point-of-care software application (i.e., the POC application 402) that may be capable of contextually calling for and integrating the suspect condition data 305 with existing health information of the EMR 212 (e.g., the health information 239 of FIG. 2), for example during a native encounter 106 between the patient 102 and the clinician 104 such as an in-person appointment, to aid the clinician 104 in evaluating the suspect condition to result in a diagnosis. A documentation server 500 may assess the documentation of the diagnosed condition to ensure compliance with one or more documentation requirements 509.

In the embodiment of FIG. 1, a patient 102 engages in a native encounter 106 with a clinician 104. The native encounter 106 is an encounter with live communication between the patient 102 and clinician 104. The native encounter 106 may, for example, be an in-person appointment, a teleconference, a live chat session or text messaging exchange, and/or a video conference. The patient 102 may engage in the native encounter 106 for any number of health-related reasons, for example, a general check-up or prescription refill, to have a symptom evaluated, and/or to have a health condition evaluated. The clinician 104 utilizes a computing device 400, such as a desktop computer, laptop computer, tablet computer, and/or smartphone, to evaluate the patient 102 using the POC application 402. The POC application 402 may provide a wide array of functionality, for example retrieving and reviewing health information from the EMR 212 of the patient 102 (e.g., the health information 239 of FIG. 2), accessing resource directories of physicians within a healthcare network, etc. The POC application 402 may provide a clinical documentation workflow 406 that includes a workflow for evaluating, diagnosing, documenting, creating care programs for, and otherwise addressing aspects of a health condition. The computing device 400 and/or the POC application 402 may provide the clinician 104 with a user interface 411 for viewing data of the EMR, manipulating the clinical documentation workflow 406, and other functions.

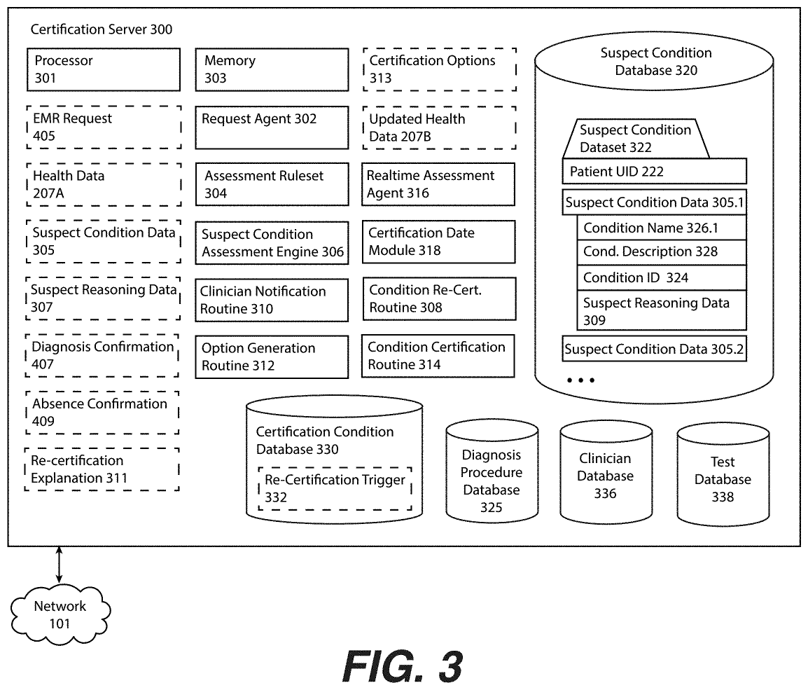

Prior to and/or during the native encounter 106, the certification server 300 may evaluate the EMR 212 of the patient 102 for one or more suspect conditions. Data specifying the suspect conditions (i.e., the suspect condition data 305) may be served to the clinician 104 by the certification network 100 such that it promotes efficient and/or accurate certification as a diagnosed condition, as shown and described in the present embodiments. The certification server 300 calls for a health data 207 which is extracted from the EMR 212. An assessment ruleset 304 is applied to the health data 207 by a suspect condition assessment engine 306 and/or a re-certification condition routine 308 that evaluates the health data 207 against a re-certification trigger (e.g., the re-certification trigger 332 of FIG. 3). The output may be one or more instances of the suspect condition data 305. In what may be a simple example, a previously recorded instance of a body mass index (B MI) score in the health data 207 may be determined to exceed a reference value such that obesity is currently likely in the patient 102. In another example, where the patient 102 previously experienced a crippling injury to a limb over one year ago, and it is possible such injury has persisted, a re-certification trigger may determine the injury is a suspect condition for re-certification. The one or more suspect condition data 305 may be stored on the certification server 300, the EMR database 210 (for example in a partition of the EMR 212 of the patient 102 so no confusion arises as to a certified versus non-certified condition), and/or in another location.

During the native encounter 106 between the patient 102 and the clinician 104, the POC application generates an EMR request 405 for health information of the patient 102 to be read from the EMR 212 by the EMR management application 204. The health information (i.e., the health information 239 of FIG. 2) may include any information stored within the EMR 212, for example charts, test results, medical images, medical graphs, logs, notes, and/or other health information. The EMR request 405 may be generated automatically as a routine aspect of the native encounter 106 or may be in response to the clinician 104 calling for specific information for a specific purpose. The native encounter 106 may also be logged by an audit log routine 206 in the EMR 212, where such audit log (e.g., the audit log 224 of FIG. 2) can be used to distinguish the nature of the EMR request 405 as an active, live encounter between the patient 102 and the clinician 104.

The certification server 300 detects the EMR request 405, may further detect the EMR request 405 is within the native encounter 106 (e.g., by assessing the audit log), and initiates a clinician notification routine 310. The clinician notification routine 310 transmits the one or more instances of the suspect condition data 305 through the network 101 to the computing device 400.

The computing device 400 organizes the health information 239 and the suspect condition data 305 in a visual association on a user interface of the POC application 402 within the clinical documentation workflow 406. The organization may be defined and/or selected to maximize relevant information extracted from the EMR 212 for evaluating the suspect condition data 305 for diagnosis. The visual coordination of the suspect condition data 305 relative to existing information extracted from the EMR, simultaneously during the native encounter 106 with the patient 102 in which additional information may be easy to obtain, may collectively provide for both an efficient and accurate process for certifying a suspect condition.

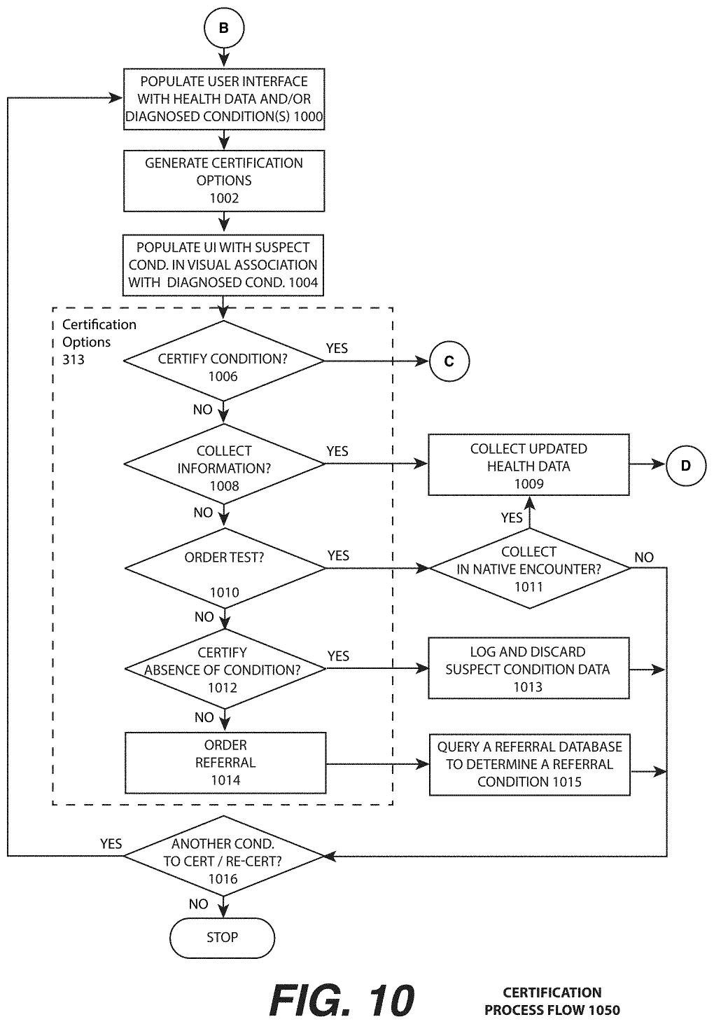

In addition to a visual association, a number of certification options 313 (e.g., as shown and described in conjunction with FIG. 3) may be formulated for the suspect condition data 305 to aid the clinician in efficient and/or accurate diagnosis. For example, as shown and described in conjunction with FIG. 3 and FIG. 10, the certification options 313 may include: (i) certifying the condition, (ii) certifying absence of the condition, (iii) ordering and/or administering a test, (iv) collecting additional information (that may not qualify as a formal test but may still assist in diagnosis), and/or (v) ordering a referral to another clinician. Choices relevant for the suspect condition may be determined and associated with the certification option. For example, to diagnose a skin condition a list of qualified dermatologists (e.g., in a network of the healthcare provider) may be read from a directory and made available, for example through a drop-down menu on the user interface of the POC application 402.

Following evaluation of the suspect condition, for example by both reviewing the patient 102 and/or the health information 239 of the EMR 212 of the patient 102, the clinician 104 may diagnose the suspect condition as a diagnosed condition by certifying its presence with the certification option 313. The diagnosis confirmation 407 is generated by the computing device 400, which is transmitted through the network 101 to the record server 200 and/or the certification server 300. The diagnosis confirmation 407 initiates the entry of new information in the EMR 212 through a record creation instruction. Where the newly diagnosed condition is a new health condition, a condition record 226 may be created and the suspect condition data 305 of the newly diagnosed condition integrated into the condition record 226. Where the suspect condition was a previously diagnosed condition that has been re-certified, an existing condition record 226 may be maintained while a certification date 234 may be updated and/or a log of the new certification date 234 added.

The documentation server 500 may utilize a documentation assessment engine 502 to reference a documentation requirement database 506 for documentation requirements of the new instance of the diagnosed condition. The requirement may be the result, for example, of an internal quality control, an insurance claim requirement, a contractual requirement, and/or a government regulation. In a more specific example, the internal quality control may be a prescribed diagnosis test promulgated by a non-governmental organization such as the American Dental Association. The documentation assessment may occur prior to certification and/or re-certification of the suspect condition as a diagnosed condition. For example, within the user interface 411 of the POC application 402 a flag or alert may appear if available information in the EMR 212 is insufficient to document the condition. While the native encounter 106 is still ongoing, the clinician 104 may be alerted to the documentation deficiency so that the documentation can either be obtained immediately or so that any tests or follow-up appointments can be setup. Documentation may be efficiently collected, and may be of increased accuracy, as a result of collection within the native encounter 106 and close in time to the certification of the health condition which is being documented.

In the embodiment of FIG. 1, the a healthcare professional qualified and permitted to diagnose and certify the suspect condition as existing in the patient. The network 101 connecting each of the elements of the certification network 100 may be a communications network such as a virtual private network (VPN), a local area network (LAN), a wide area network (WAN), and/or the Internet. In one or more embodiments a network 101A may be a VPN or LAN, which may be connected to the network 101B which may be a WAN or the internet.

FIG. 2 illustrates the record server 200 of FIG. 1, including an EMR management application 204, an audit log routine 206 for logging a native encounter 106 between the patient 102 and the clinician 104, and the EMR 212 of the patient 102 comprising one or more instances of a condition record 226 of a diagnosed condition and optionally a partition 240 in which one or more instances of the suspect condition data 305 may be stored, according to one or more embodiments.

The record server 200 includes a computer processor (i.e., the processor 201) and a computer memory (i.e., the memory 203). An authentication module 202 comprises computer readable instructions that when executed on the processor 201 authenticates the clinician 104 and/or the computing device 400. Authentication may be based on one, two, or three authentication factors (e.g., something the clinician knows such as a password, something the clinician 104 has in possession such as a fob, and/or something the clinician 104 “is”, such as a biometric like a thumb print). Authentication of the clinician 104 and/or the computing device 400 associated with the clinician 104 may be important for ensuring an authorized and/or qualified certification of a diagnosed condition to ensure accuracy in the EMR 212.

An EMR management application 204 comprises software for managing, storing, extracting, querying, encrypting, and backing up, electronic data in an EMR database 210. The EMR management application 204, for example, may be EPIC, Cerner, McKesson, All Scripts, MedSys. The EMR management application 204 may extract information of the EMR 212 in response to a query and generate and/or modify a condition record 226 in response to a record creation instruction that may be issued to an application programming interface (API) of the EMR management application 204. The audit log routine 206 is software comprising instructions that records and/or store audit logs of health events related to the patient 102. For example, the audit log routine 206 may comprise computer readable instructions that when executed on the processor 201 of the record server 200 generate an audit log 224 comprising data specifying that the patient 102 is in the presence of the clinician 104, e.g., to document the native encounter 106. The partitioned storage routine 208 is software for storing uncertified data in a physical separation from the EMR 212. For example, prior to certification the suspect condition data 305 may be stored in a separate database and/or in a distinct partition from the EMR 212 to ensure diagnosis accuracy and/or data integrity. The partitioned storage routine 208 may comprise computer readable instructions that when executed on the processor 201 generate a request for the EMR management application 204 to store the suspect condition data 305 in a partition 240 that is a database partition distinct from the one or more diagnosed conditions of the EMR 212 (e.g., each of the diagnosed conditions may be stored in a condition record 226).

The record server 200 comprises an EMR database 210 that stores one or more instances of the EMR 212. The EMR database 210 may utilize one or more data models to store data (e.g., a relational database, a key-value store, a graph database, a columnar database). The EMR database 210 may be implemented with a commercial database such as Oracle, MySQL, Inter Systems Cache. The EMR 212 is a set of electronically stored medical records of a patient 102. The EMR 212.1 comprises a number of data attributes that may have associated data values. A patient UID 222 is a unique identifier usable to uniquely identify the patient and/or the EMR 212 of the patient. The patient UID 222 may be, for example, a medical record number, a clinical record number, and/or a record set ID of the EMR 212. An audit log 224 stores data of one or more logs related to health events of the patient 102, including but not limited to documenting the native encounter 106. The audit log 224 may be stored in an audit database 250, which may include the patient UID 222.

The EMR 212 may have one or more instances of a condition record 226.1 through a condition record 226.n. Each condition record 226 is data specifying a condition for which a certified diagnosis has occurred. The condition record 226 may include a condition name 228 (which may be a common name, a scientific name, and/or a technical name) and a description 230. The condition record 226 may include a condition ID 232 that is an identifier of the health condition specified by the condition record 226. For example, the condition ID may be a hierarchical condition code (HCC) and/or a diagnosis code.

A quantitative test result 216 may include data specifying a type of quantitative test undertaken by the patient 102 along with the result of the quantitative test. For example, the quantitative test result may comprise data from a physical characteristic of the patient, a vital sign, and a laboratory test. The laboratory test may be based on biological, microbiological, serological, chemical, immunohematological, hematological, biophysical, cytological, pathological, or other examination of materials derived from the human body for the purpose of providing information for the diagnosis, prevention, or treatment of any disease of the patient 102. The quantitative test result 218 may be generated in a standard communication protocol such as the Fast Healthcare Interoperability Resources (FHIR) standard and/or the HL7 standard.

The qualitative test result 218 may include data specifying a type of qualitative test undertaken by the patient 102 along with the result of the qualitative test. The qualitative test result 218 may include data from family history evaluation, a patient generated report, a system review, a verbal screening, a psychiatric evaluation, a medical imaging data, and/or a medical graph data. The medical imaging data may include images such as X-ray radiography, ultrasound, computed tomography (CT), nuclear medicine including positron emission tomography (PET), and/or magnetic resonance imaging (MRI). The condition record 226 comprises one or more instances of a certification date 234.1 through a certification date 234.n, specifying date(s) and/or times during which the condition was certified. The certification date 234.1 may also be stored in association with, stored within, and/or referenced by a relevant instance of the audit log 224.

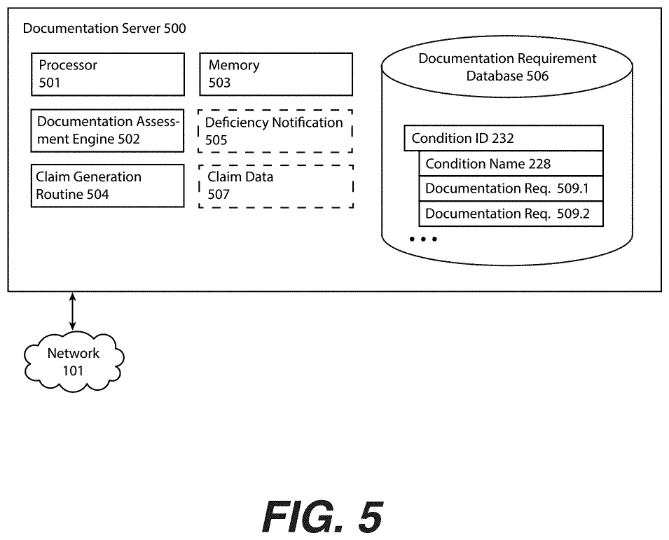

One or more instances of the document 236 may store other data of the EMR 212, for example clinician notes or scanned documents. The document 236 may be assessed for a documentation requirement (e.g., the documentation requirement 509 of FIG. 5). Although shown as separate fields in the embodiment of FIG. 2, the document 236 may also include the quantitative test result 216 and/or the qualitative test result 218.

The partition 240 may be a segregated location for storing data within the EMR database 210 such that there is a reduced risk the suspect condition data 305, which may have not gone through a formal certification, could be confused with a condition record 226 or other confirmed heath data of the EMR database 210. The patient UID 222 may be used to create an association between the EMR 212 and the partition 240 without incorporating the suspect condition data 305 into the EMR 212.

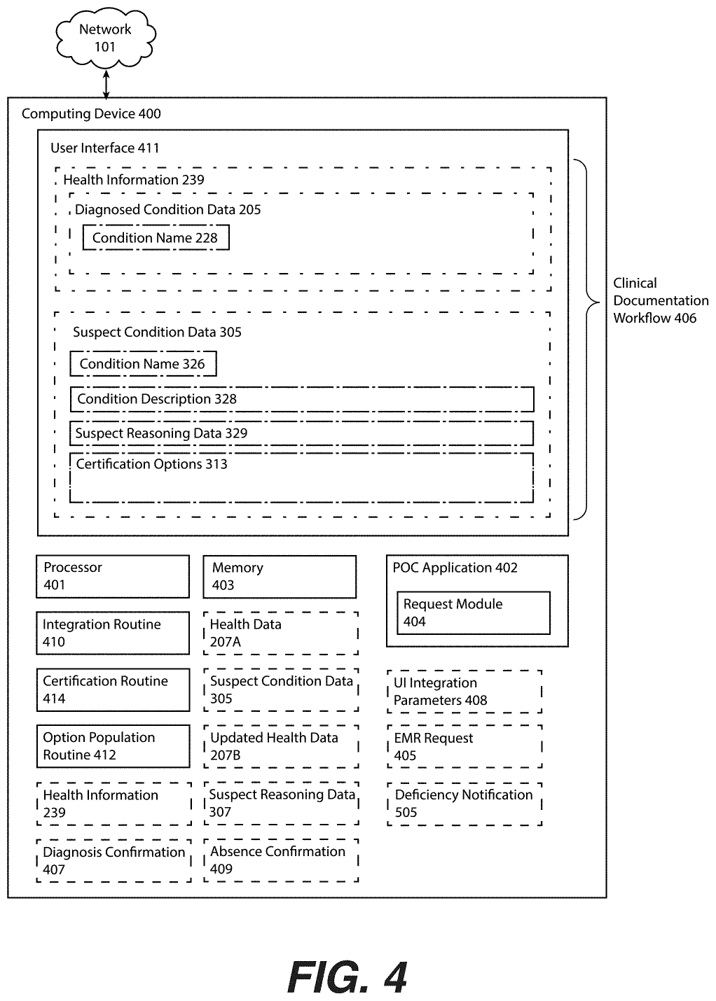

The record server 200 may comprise a number of stored instances of data. The health data 207 may be data extracted from the EMR 212 for the purposes of analyzing for suspect conditions. The record server 200 may receive and temporarily store the EMR request 405, as shown and described in FIG. 4. The health data 207 is data extracted from the EMR 212 for the purpose of identifying suspect conditions. The health data 207 is illustrated in a first state as the health data 207A, and in a second state as the updated health data 207B which may be supplemented by additional data input during the native encounter 106. The health data 207 may include the quantitative test result 216, the qualitative test result 218, and/or a diagnosed condition data 205. The diagnosed condition data 205 may be data extracted from the EMR 212 representing some or all of the condition record 226. For example, the diagnosed condition data 205 may include the condition name 228, the condition ID 232 such as an HCC. Similarly, the health information 239 may also be extracted from the EMR database 210 but for the purpose of presenting the EMR 212 to the clinician 104. Where a document 236 may be relevant to include in the health data 207, it may be irrelevant within the health information 239 unless explicitly requested by the clinician 104. The record server 200 may store the suspect condition data 305, as shown and described in conjunction with the embodiment of FIG. 3. Both the health data 207 and the health information 239 may be extracted through the EMR management application 204 in response to an EMR request 405 over the network 101.

FIG. 3 illustrates the certification server 300 of FIG. 1, including a suspect condition assessment engine 306 that applies an assessment ruleset 304 to health data 207 extracted from the EMR database 210 of FIG. 2 to generate a suspect condition, the certification server 300 possibly comprising a suspect condition database 320 for storing the suspect condition (e.g., as a suspect condition data 305), and a certification routine 314 for certifying the diagnosis of the suspect condition to generate a record in the EMR 212 (e.g., a new instance of the condition record 226 of FIG. 3), according to one or more embodiments.

The certification server 300 includes a computer processor (i.e., the processor 301) and a computer memory (i.e., the memory 303). A request agent 302 comprises computer readable instructions that when executed on the processor 301 detects the EMR request 405. The EMR request 304 may have been generated by a POC application 402 running on a computing device 400 of a clinician 104. The EMR request 304 may be a request specifying a retrieval instruction for information to be extracted from the electronic medical record (e.g., the health information 239) of the patient 102. The request agent 302 may alternatively or in addition execute on the record server 200. The request agent 302 may call the clinician notification routine 310.

The assessment ruleset 304 is a set of one or more rules that outputs a suspect condition based on the health data 207, where the suspect condition is a potential health condition diagnosable by the clinician 104. The assessment ruleset 304 may be comprised of if/then statements, an analysis algorithm generating risk factor scores, and/or utilize other automatic methods of outputting the suspect health condition. For example, the assessment ruleset 304 may specify a guideline of the American Heart Association that if systolic blood pressure is greater than 140 mm Hg or diastolic blood pressure is greater than 90 mm Hg then the patient 102 may have a suspected condition of stage 2 hypertension (e.g., IF (SysBlood>140 OR DiaBlood>90) THEN Suspect=HyperTension2). In another example, if a word such as “forgetful” or “disoriented” appears in a clinician note in the EMR 212 and the patient 102 is over the age of 65 years, then a suspected condition may be dementia. In yet another example, if the patient 102 resides within three miles of a known or possible contamination site specified in a database (e.g., poor air quality, a chemical spill, and/or a facility that handles radioactive materials) and experiences certain symptoms associated with the contamination a suspect condition may be generated.

The suspect condition assessment engine 306 generates data specifying a suspect condition, referred to as the suspect condition data 305. The suspect condition assessment engine 306 may comprise computer readable instructions that when executed on the processor 301 request the health data 207 of the patient 102 from the EMR 212 of the patient 102. The health data 207 may include one or more diagnosed conditions (e.g., stored as the diagnosed condition data 205), the quantitative test result 216, and/or the qualitative test result 218. The computer readable instructions may apply the assessment ruleset 304 to the health data 207 of the patient 102 that outputs a suspect condition based on the health data 207. The computer readable instructions may also generate a suspect condition data 305 comprising a name of the suspect condition. The suspect condition assessment engine 306 may also receive a suspect condition and/or formulate a suspect condition data 305 from an external system, for example through an API over the network 101. Following generation and/or receipt of one or more instances of the suspect condition data 305, the suspect condition assessment engine 306 may store the one or more instances in the suspect condition database 320.

The suspect condition data 305 comprises data specifying the suspect condition of the patient 102, and may include additional data that may be helpful in clarifying or creating context for the clinician 104 and/or include data usable to create a new record in the EMR 212 if the suspect condition is certified as a diagnosed condition. For example, the suspect condition data 305 may comprise a name of the suspect condition (i.e., the condition name 326), a description of the suspect condition (i.e., the condition description 328, a suspect reasoning data 307, and/or condition ID 324 (e.g., a hierarchical condition category code).

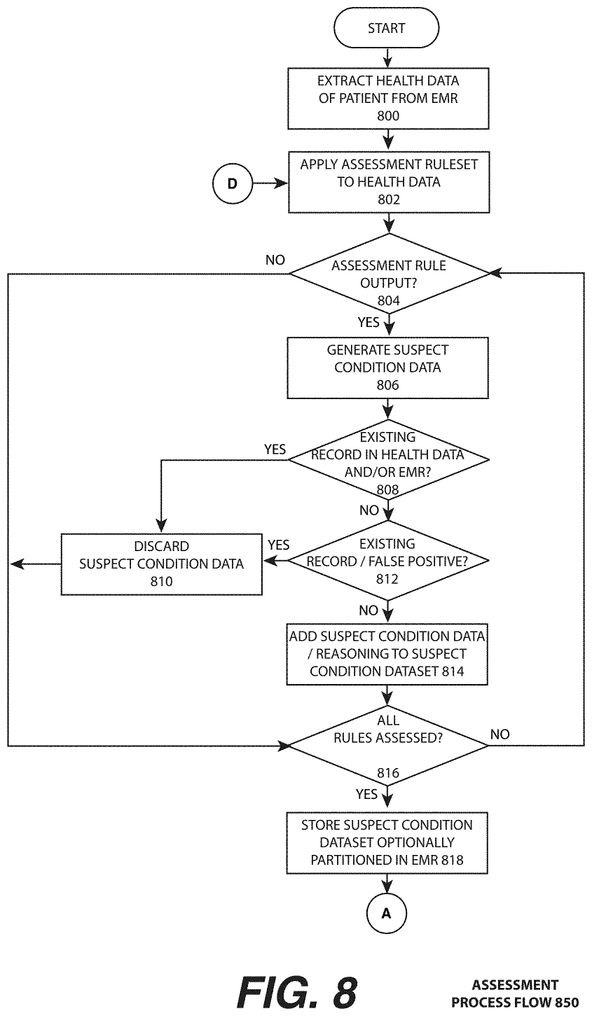

The suspect condition assessment engine 306 may comprise computer readable instructions that when executed on the processor 301 determine that the suspect condition is a care gap of the patient through comparison to the health data 207 and/or the one or more diagnosed conditions in the EMR 212 (e.g., comparison to data of condition record 226). For example, as shown and described in the embodiment of FIG. 8, a false positive may be detected by determining that the suspect condition data 305 is already a diagnosed condition stored in a condition record 226 of the EMR database 210 (e.g., by comparison of an HCC code of the suspect condition and an HCC code of the diagnosed condition of the condition record 226). The suspect condition assessment engine 306 may comprise computer readable instructions that when executed on the processor 301 generate a suspect reasoning data 307 specifying one or more reasons the suspect condition was output from application of the assessment ruleset 304 to the health data 207 of the patient 102. For example, the suspect reasoning data 307 may translate one or more rules of the assessment ruleset 304 into a narrative, or human-readable text, that may be easily understood when presented to the clinician 104. For example, a rule of “IF (SysBlood>140 OR DiaBlood >90) THEN Suspect=HyperTension2” that leads to a suspect condition result may be converted to: “The patient had a systolic blood pressure of 142, and a diastolic blood pressure of 88. Based on systolic blood pressure, the patient may be classified as Class 2 hypertension.” The suspect reasoning data 307 may also be stored and retrieved through a link or menu item if required by the clinician, as shown and described below.

The suspect condition may be stored as a suspect condition data 305 in a suspect condition dataset 322. The suspect condition dataset 322 comprises one or more instances of the suspect condition data 305 which may have originated, for example, from evaluation of one or more instances of the re-certification trigger 332 and/or the application of an assessment ruleset 304. The suspect condition dataset 322 may be stored for an indefinite period until called, for example for presentation on a computing device 400.

A clinician notification routine 310 comprises computer readable instructions that when executed on the processor 301 transmits one or more instances of the suspect condition data 305 in coordination with information of the EMR 212 to the computing device 400 over the network 101. The clinician notification routine 310 may extract the condition data 305 from the suspect condition dataset 322. The information of the EMR 212 may include one or more diagnosed conditions for presentation to the clinician 104 through a user interface of the POC application 402 within the clinical documentation workflow 406. The transmission may be in response to and/or initiated by a call from the request agent 302. The health information 239 and the suspect condition data 305 may be transmitted together in a single communication over the network 101, in one or more embodiments.

The option generation routine 312 may generate certification options 313 that may also be transmitted to the computing device 400. The option generation routine 312 may generate options comprising one or more of a diagnosis confirmation option, an absence confirmation option, a test order option, a referral order option, and an information collection option. The diagnosis confirmation option initiates generation of a diagnosis confirmation (e.g., the diagnosis confirmation 407 as shown and described in FIG. 4) that in turn may generate an instruction to create a new record in the EMR 212, and may create an associated instance of the audit log 224 (e.g., in the audit database 250). The absence confirmation option initiates generation of an absence confirmation (e.g., the absence confirmation 409 as shown and described in FIG. 4) that in turn may generate an absence record certifying that the suspect condition is absent from the patient 102, for example by discarding the suspect condition data 305 and logging the event in an audit log 224.

A referral order option initiates a referral process to refer the patient 102 to a different instance of the clinician 104, which may be referred to as a referral clinician, where the referral clinician is qualified to diagnose the suspect condition. Options for the referral clinician may be automatically generated in one or more embodiments, for example by extracting the condition ID 324 from the suspect condition data 305 and calling a clinician database 336 with the condition ID 324 to return one or more of the referral clinicians qualified to diagnose and/or certify the suspect condition. The test order option may initiate ordering a diagnostic test approved to diagnose the suspect condition represented by the suspect condition data 305. In what may have some similarity to the automatic determination of the qualified referral clinician, the diagnostic test(s) that may be approved can be determined and extracted using the condition ID 324 or other means from a test database 338. The option generation routine 312 may prioritize tests in terms of cost, known accuracy in determining a correct diagnosis, etc. The test may be a type of test that can be administered to the patient at the native encounter 106, at a later native encounter 106, and/or administered by another entity or party (e.g., a laboratory test the patient 102 must report to a laboratory to receive). An information collection option may initiate a data collection process (e.g., other than a formal test) that gathers additional information usable to diagnose the suspect condition within the native encounter 106. For example, the information collection option may initiate an electronic notepad for the clinician to take notes that may be further analyzed, may initiate download of health data under the control of the patient (such as on a device like a smartwatch or pacemaker), and/or permit recording of a narrative of the patient that may be converted via speech-to-text software and assessed. Both new test results and/or new information received may be referred to as the updated health data 207B, which may or may not be added to the EMR 212. Finally, a diagnosis procedure option may retrieve a diagnosis procedure and/or a guideline from a diagnosis procedure database 325 for the clinician 104 to review, which may include one or more links to the test database 338. The test order option and the information collection option are further shown and described in conjunction with the embodiment of FIG. 8.

The certification options 313 may be standard for all suspect conditions, may be customized for a certain class of the suspect condition, and/or may be uniquely generated for a suspect condition. For example, in a non-limiting example, a certain rare health condition may only be able to be certified by one or a few specialists, and the clinician 104 may be required to refer the suspect condition.

A condition certification routine 314 receives and/or generates a diagnosis confirmation 407 and/or an absence confirmation 409 and makes appropriate calls to, respectively, record the diagnosis confirmation 407 as a condition record 226 in the EMR 212 or discard the suspect condition data 305. The condition certification routine 314 may comprise computer readable instructions that when executed on the processor 301 generate a diagnosis confirmation 407 of the suspect condition to certify and/or re-certify the diagnosed condition of the diagnosed condition data 205. The condition certification routine 314 may also comprise computer readable instructions that when executed on the processor 301 generate a record creation instruction (e.g., for the EMR management application 204) for creation of a new record (e.g., a condition record 226) in the EMR 212 of the patient 102.

In addition to suspect conditions generated from application of the assessment ruleset 304, suspect conditions may also be generated through evaluation of one or more re-certification conditions that may be referred to as a re-certification trigger 332. The re-certification trigger 332 may be stored in a certification condition database 330. The re-certification trigger 332 may apply generally to many types of health conditions (e.g., a chronic condition that must be certified annually) and/or may be specified for a certain health condition (e.g., a new standard is required for a patient to qualify as having a health arrhythmia and therefore the existing condition record 226 may represent a suspect condition under the new standard requiring re-certification).

The condition re-certification routine 308 may comprise computer readable instructions that when executed on the processor 301 determine occurrence of a re-certification trigger 332 with respect to a condition record 226 of the EMR 212 of the patient 102. The re-certification condition routine 308 may generate a re-certification explanation 311, that, in what may have similarity to the suspect reasoning data 307, specifies one or more reasons why what is now the suspect condition was subject to the re-certification trigger 332. For example, the re-certification explanation 311 may specify that a diagnosis standard has changed. The re-certification condition routine 308 may comprise computer readable instructions that when executed on the processor 301 generate a suspect condition data 305 comprising a name of the suspect condition (i.e., the condition name 326), a description of the suspect condition (i.e., the condition description 328), and condition ID 324 such as a hierarchical condition category code. The computer readable instructions may further, when executed, transmit the suspect condition data 305 in coordination with information of the EMR 212 (e.g., the health information 239) from a server to the computing device 400 over the network 101. The information of the EMR 212 may include one or more diagnosed conditions and may be presented to the clinician 104 through a user interface 411 of the POC application 402 within the clinical documentation workflow 406. The diagnosed condition may be represented by a diagnosed condition data 205.

A certification date module 318 processes a diagnosis confirmation 407 to add and/or update the certification date 234 of a condition record 226. The certification date module 318 may comprise computer readable instructions that when executed on the processor 301 adds a new certification date 234 associated with the existing instance of the condition record 226.

The certification server 300 may also include a realtime assessment agent 316. The realtime assessment agent 316 may determine updated information is available which may form the basis of generating a suspect condition data 305. For example, the realtime assessment agent 316 may detect the updated health data 207B generated as a result of the diagnostic test result and/or the information collection process of the certification options 313. However, in one or more embodiments, the realtime assessment agent 316 may also detect data added to the EMR 212 during the native encounter 106 but which may have been added by another clinician 104 (e.g., in another location), or that a change in a diagnosis standard was just recorded in data. The realtime assessment agent 316 may also determine an updated instance of the assessment ruleset 304 may be available for re-application to the health data 207.

The realtime assessment agent 316 may comprise computer readable instructions that when executed on the processor 301 detect an updated instance of the health data 207 (e.g., the updated health data 207B) and/or an update to the EMR 212 and calls the suspect condition assessment engine 306 for application of the assessment ruleset 304 to the updated health data 207B and/or optionally the health data 207A.

FIG. 4 illustrates the computing device 400 of FIG. 1 running the POC application 402 aiding the clinician 104 in diagnosing the patient, the computing device 400 receiving health information 239 from the EMR 212 of FIG. 2 and the suspect condition data 305 generated by the certification server 300 of FIG. 3, and integrating them both along with one or more certification options 313 in a clinical documentation workflow 406 to assist in certifying the suspect condition, according to one or more embodiments.

The computing device 400 comprises a computer processor (i.e., the processor 401) and a computer memory (i.e., the memory 403). The computing device 400 includes a display (e.g., an LCD screen) capable of presenting a user interface 411. The computing device 400 may additionally have a speaker and microphone. In one or more embodiments, the computing device 400 may be a desktop computer, a computing workstation, a laptop, a notebook computer, a tablet computer and/or a smartphone.

The POC application 402 is a point-of-care software application capable of retrieving, displaying, and receiving instructions to modify data of the EMR 212. The POC application 402 may be a commercially available point-of-care software. The POC application 402 comprises a request module 404 for generating the EMR request 405 for retrieval of the health information 239 from the EMR 212 over the network 101. The request generated by the request module 404 may be detected by the request agent 302, as shown and described in FIG. 3. The POC application 402 may further comprise UI integration parameters 408 for specifying visual relationships within the user interface 411. The UI integration parameters 408 may include data parameters for sizing, formatting, and/or organization of a visual element (such as a window, text box, and/or image), one one visual element relative to another, and any other display parameters of the user interface 411. The UI integration parameters 408 may be referenced for organizing the clinical documentation workflow 406. In one or more embodiments, a visual element may be a window presenting the suspect condition data 305 and/or the diagnosed condition data 205.

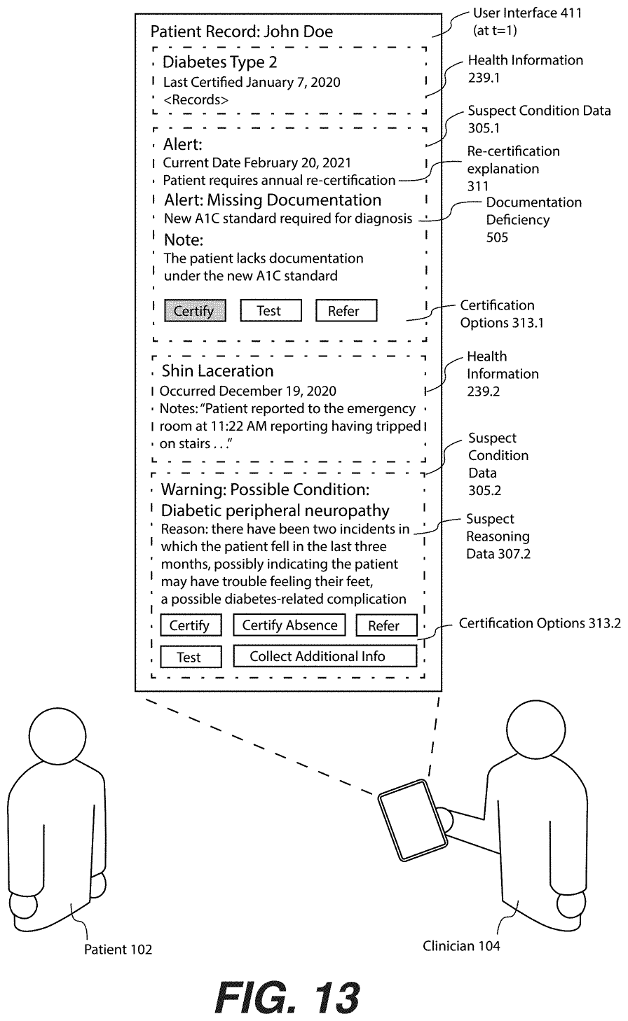

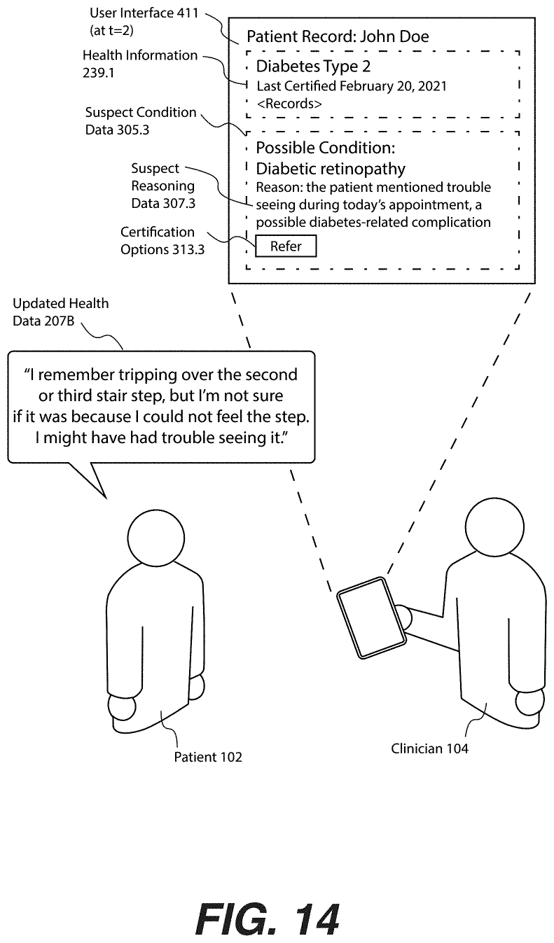

Data presented to establish context of the clinician 104 may be the health information 239 of the patient 102, which may include the diagnosed condition data 205. The suspect condition data 305 presented may include the condition name 326, the condition description 328, a suspect reasoning data 329, and one or more certification options 313. A few examples of presentation of the health information 239 and the suspect condition data 305 are shown and described in the example embodiments of FIG. 13 and FIG. 14.

An integration routine 410 comprising computer readable instructions that when executed on the processor 401 integrate the suspect condition data 305 in visual association with the health information 239, such as one or more diagnosed conditions extracted from the EMR 212 (e.g., as the diagnosed condition data 205) within the clinical documentation workflow 406 to provide a context for the suspect condition relative to the one or more diagnosed conditions within a native encounter 106 between the clinician 104 and the patient 102. The integration routine 410 may read and/or edit the UI integration parameters 408 to determine a visual association of a visual element containing the suspect condition data 305 and a visual element containing the diagnosed condition data 205. The integration routine 410 may additionally comprise computer readable instructions that when executed on the processor 401 integrate the suspect reasoning data 307 in visual association with the health information 239, such as one or more diagnosed conditions extracted from the EMR 212 as diagnosed condition data 205.

An option population routine 412 generates and/or receives menu options to be visually associated with the suspect condition data 305 on the user interface 411. In one or more embodiments, the option population routine 412 may receive a set of certification options 313 which may be determined and/or generated by the option generation routine 312. For example, the option population routine 412 may receive data specifying relevant certification options 313 for each instance of the suspect condition data, e.g., a diagnosis confirmation option, an absence confirmation option, and a referral order option, including communicating data specifying relevant referral clinicians which may be selected by the clinician 104. In one or more embodiments, the population routine 412 comprises computer readable instructions that when executed on the processor 401 generate a set of certification options 313 in association with the suspect condition data 305 on the user interface 411, the set of certification options 313 comprising a diagnosis confirmation option and the absence confirmation option, the referral order option, the test order option, and/or the information collection option.

The certification routine 414 comprises computer readable instructions that when executed on the processor 401 processes a selection of an option of the certification options 313. In one or more embodiments, the certification routine 414 comprises computer readable instructions that when executed on the processor 401 generate a diagnosis confirmation 407 of the suspect condition to certify and/or re-certify the diagnosed condition. The diagnosis confirmation 407 may be data specifying that the diagnosis has been certified, including but not limited to data usable in the audit log 224. The diagnosis confirmation 407 may include a record creation instruction for creation of a new record (e.g., a new instance of the condition record 226) in the EMR 212 of the patient 102 upon certification of the diagnosed condition. The diagnosis confirmation 407 may be communicated along with the suspect condition data 305 and/or data of the suspect condition data 305 to the record server 200. The certification routine 414 may similarly generate the absence confirmation option 409, which may include an instruction to discard the suspect condition data 305 and/or generate an audit log 224 specifying the absence confirmation.

In one or more embodiments, the computing device 400 may collect additional health data from the patient 102 that may be assessed by the assessment ruleset 304 and/or the condition re-certification routine 308 to generate suspect conditions. The additional health data is referred to as the updated health data 207B. For example, the clinical documentation workflow 406 may be receiving inputs from the clinician 104, storing electronic notes, recording an audio narrative, and/or may have presented a qualitative test for the clinician to administer during the native encounter 106. The updated health data 207B may be communicated through the network 101 to the certification server 300 for detection by the realtime assessment agent 316.

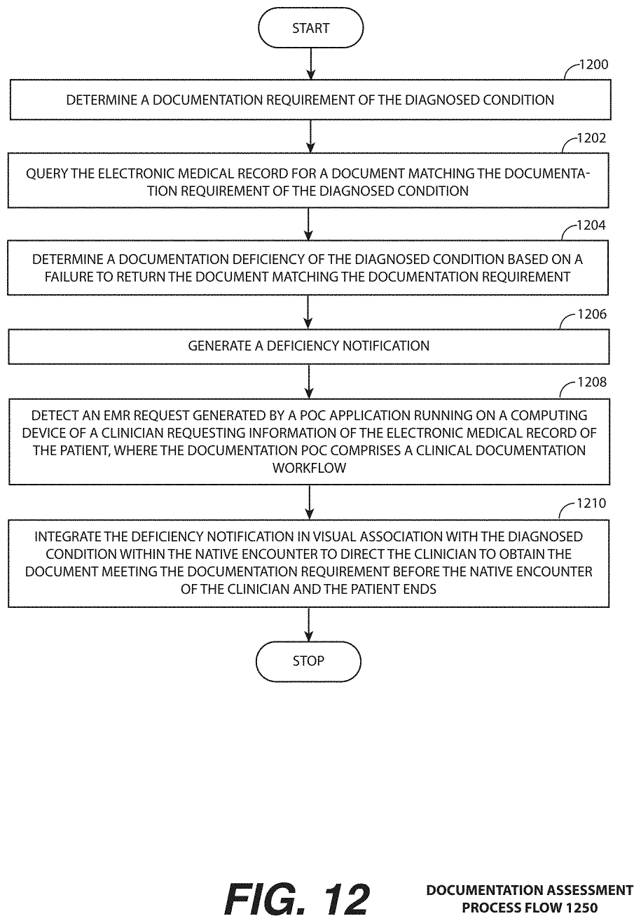

The computing device 400 and/or the POC application 402 may include a number of other functions. For example, a deficiency notification 505 may be received and displayed in response to a determination of a documentation deficiency, as shown and described in conjunction with FIG. 5 and FIG. 12.

FIG. 5 illustrates the documentation server 500 of FIG. 1 for assessing documentation of the suspect condition, according to one or more embodiments. The documentation server 500 comprises a documentation requirement database 506 associating one or more health conditions (as may be identified by a condition ID 232 and/or a condition name 228) with one or more documentation requirements 509, e.g., a documentation requirement 509.1 through a documentation requirement 509.n. A documentation assessment engine 502 evaluates documentation supporting the suspect condition before or after certification and/or recertification as a diagnosed condition and possibly generating a deficiency notification 505. A claim generation routine 504 may automatically generate an insurance claim comprising the claim data 507 for the diagnosed condition.

The documentation server 500 may include a computer processor (i.e., the processor 501) and a computer memory (i.e., the memory 503). The documentation assessment engine 502 comprises computer readable instructions that when executed on the processor of the documentation server 500 may query the documentation requirement database 506 and determine a documentation requirement 509 of the diagnosed condition. The documentation assessment engine 502 may further comprise computer readable instructions that when executed query the EMR 212 for a document (e.g., a document 236) matching the documentation requirement 509 of the diagnosed condition. The document 236 may be the quantitative test result 216 and/or the qualitative test result 218. The documentation requirement 509 may be based on data specifying at least one of an internal quality control, a new diagnosis procedure, an insurance claim requirement, and/or a government regulation.

The documentation assessment engine 502 may determine a documentation deficiency in the diagnosed condition based on a failure to return the document 236 matching the documentation requirement 509 and may further generate a deficiency notification 505 and transmit the deficiency notification 505 over the network 101, for example to the computing device 400.

The claim generation routine 504 comprises computer readable instructions that when executed on the processor 501 generate a claim data 507 that may be transmitted to a payer such as an insurance company or a government body. The claim data 507, for example, may include an insurance reimbursement claim, a claim for an insurance payment, patient data and/or another insurance-related claim information. The claim data 507 may include data of the EMR 212 and/or the suspect condition data 305 (although the suspect condition of the suspect condition data 305 may now be a diagnosed condition stored as a condition record 226 of the EMR 212).

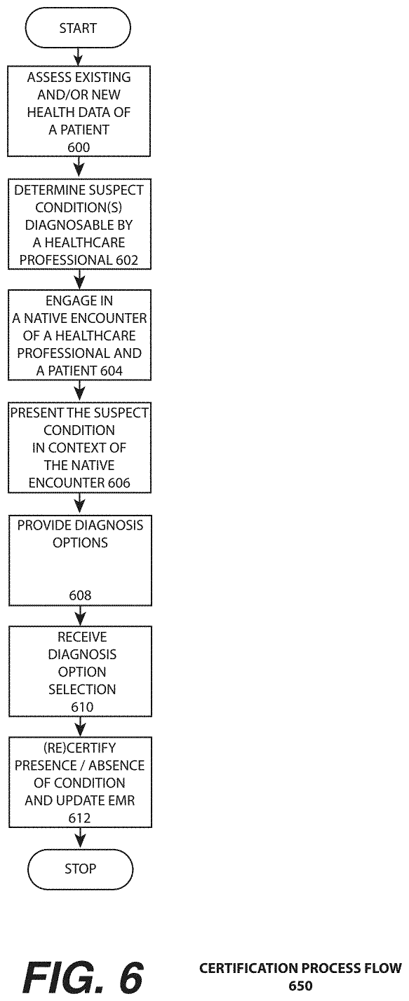

FIG. 6 is a diagnosis certification process flow 650 illustrating an overview of an accurate and/or efficient process for efficient confirmation of a suspect condition for certification and/or re-certification by a healthcare professional (e.g., the clinician 104), according to one or more embodiments. Operation 600 assesses existing health data and/or new health data of a patient 102. For example, the health data may be extracted from an electronic medical record (the EMR 212), downloaded from patient-controlled data (e.g., from a device of the patient, from a database controlled by the patient, from a distributed ledger technology (DLT) that may store encrypted patient data in a blockchain data structure, etc.), received from a third-party healthcare provider, and/or lifestyle information that may be mined or assessed for healthcare purposes (e.g., occupational data, location data, etc.). Step 602 determines one or more suspect conditions from the health data 207. A number of statistical means known in the art may be used to generate the suspect condition, for example statistical analysis of a probability the patient presently has a health condition based on many factors in the health data 207. For example, the suspect condition may be based on location data or occupational data in combination with existing data of the EMR 212. The health condition may be determined to be a condition which was previously certified by the patient 102 for which a period of time has passed such that it is unknown whether the health condition is persisting in the patient and/or a regulation, rule, or internal quality control requires re-certification by a qualified healthcare professional. The health condition is one diagnosable by a healthcare professional.

In step 604, a healthcare professional and a patient 102 engage in a native encounter 106. The native encounter 106 is a live interaction of the patient 102 and the healthcare professional (e.g., the clinician 104) that may provide context and enable and/or permit rapid, accurate, and/or efficient diagnosis of suspect condition to convert it to a diagnosed condition. For example, the native encounter may be an in-person appointment, a video conference in which the healthcare professional may see the patient 102 through a video feed and/or the patient 102 may see the healthcare professional through a video feed, a text chat which may include a capability to send images, and/or a teleconference. In step 606, the suspect condition is presented to the healthcare professional within the context of the native encounter 106 on a software application running on a computing device, for example through a point-of-care application (i.e., the POC application 402). In another example, the suspect condition may be presented to the healthcare professional within the communication medium utilized to engage in the native encounter 106. For example, where the healthcare professional is communicating with the patient 102 through a text chat, a message within the text chat may alert the healthcare professional (but possibly not the patient 102) to the suspect condition.

Step 608 provides diagnosis options to the healthcare professional for the suspect condition. For example, the certification options 313 may include confirming a diagnosis, delaying and/or ignoring a current suspect condition, confirming absence of a diagnosis, ordering a test (which may include a test that can be administered during the native encounter 106), referring the patient 102 to another healthcare professional, and/or collecting additional information that may be usable to diagnose the suspect condition. The options may be menu options integrated with presentation of the suspect condition. Step 610 receives a selection of one or more of the diagnosis options of step 608 and processes a response. For example, a certification option and/or an absence certification option may formulate data sufficient to create and/or modify a record in the EMR 212 certifying presence and/or absence of the suspect condition, respectively.

Step 612 certifies the presence or absence and/or re-certifies the presence of the suspect condition as a diagnosed condition and updates the EMR 212 of the patient 102. In step 612 data is collected sufficient to certify the diagnosis, under an internal quality control, standard, regulation, or other prescribed requirement. Such data may be logged in logs stored in the EMR 212 and/or in another database.

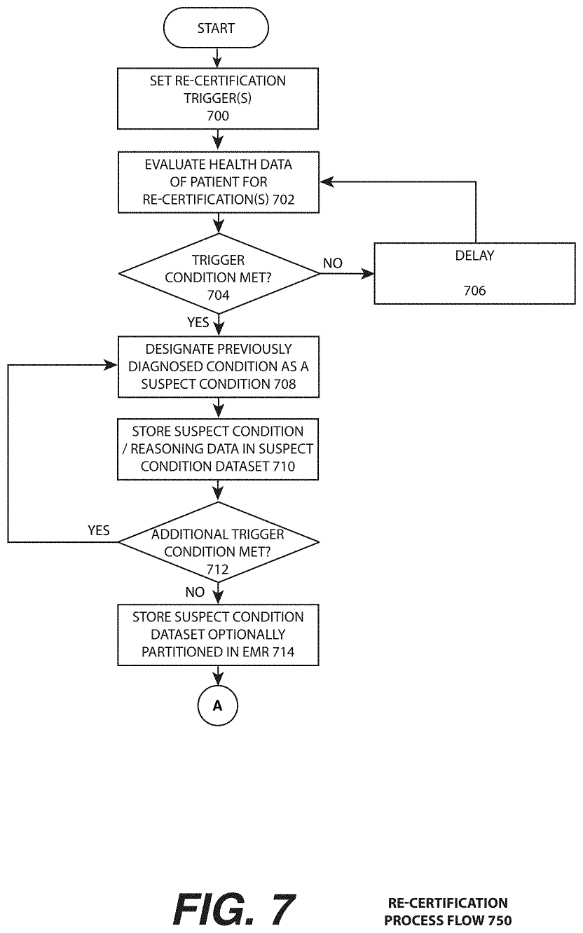

FIG. 7 is a re-certification process flow 750 illustrating generation of a suspect condition data 305 upon occurrence of a trigger condition (e.g., stored as a re-certification trigger 332), for example expiration of a certification date 234 or change of a diagnosis requirement, according to one or more embodiments. Operation 700 sets one or more re-certification triggers 332. The one or more re-certification triggers 332 may be stored in a certification condition database 330. The re-certification trigger 332 is data that may specify a condition, the occurrence of which qualifies for re-evaluation an existing health condition already known and/or documented in the patient 102, for example due to a change in a diagnosis standard, passage of a period of time that may call into question whether the condition is persisting, and/or an internal standard, insurer requirement, documentation requirement, and/or regulatory requirement. The re-certification trigger 332 may be associated with a condition ID 232. Operation 700 may be a standard set of conditions periodically defined and stored by a healthcare provider, which such conditions may be applied to each EMR 212 of a patient 102 periodically and/or opportunistically (e.g., prior to an appointment where the patient 102 will engage in a native encounter 106) or as new information is added to the EMR 212.

Operation 702 evaluates the health data 207 of the patient 102 for one or more re-certification conditions. In one or more embodiments, a health condition for re-certification may be identified by determining that a condition ID 232 associated with the re-certification trigger 332 exists in the EMR 212 of the patient 102. Where the condition ID 232 exists, the re-certification trigger 332 may apply to the patient 102 and data of the EMR 212 may be extracted from the EMR 212 sufficient to evaluate the re-certification trigger 332. For example, where the certification trigger relates to a change in diagnosis procedure requiring a quantitative test result 216, operation 702 may query the EMR 212 for the quantitative test result 216. In another example, where the certification trigger requires bi-annual re-certification of the health condition, operation 702 may read a latest instance of the certification date 234. In yet another example, where the certification trigger relates to a change in an insurance requirement that a health condition is diagnosed in-person, operation 700 may read an audit log 224 associated with creation of the condition record 226 that includes the suspect condition for re-certification.

Operation 704 evaluates whether a trigger condition is met by comparing the extracted and/or read data from the EMR 212 of the patient 102 to the re-certification trigger 332. If the condition is not met, operation 704 proceeds to operation 706, which may set a delay before re-evaluation (e.g., 1 minute, 1 day, two weeks, 1 month, six months, etc.). Where the trigger condition is determined to have been met, operation 704 proceeds to operation 708. Operation 708 designates a previously diagnosed condition as a suspect condition. For example, the previously diagnosed condition may be a health condition for which a condition record 226 exists in the EMR 212. In one or more embodiments, the designation may occur without modifying the EMR 212, for example by creating a suspect condition data 305 and storing the suspect condition data 305 in the memory 303.

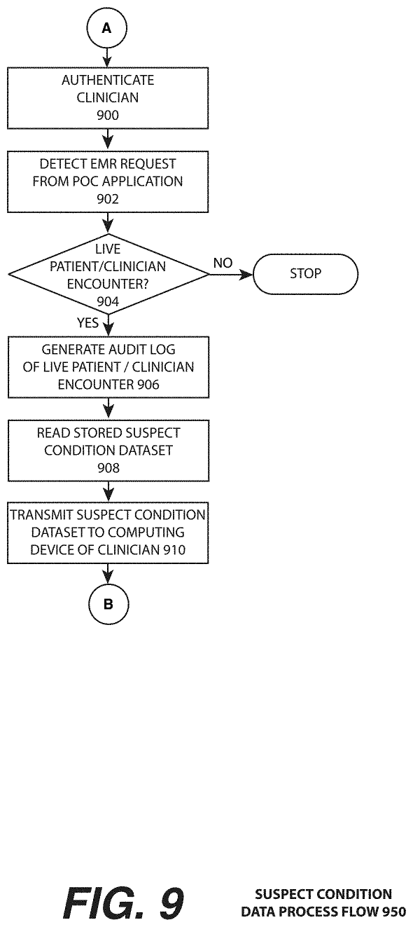

Operation 710 stores a suspect condition (as the suspect condition data 305) and/or a reasoning data in a suspect condition dataset 322 (e.g., the suspect reasoning data 309). The suspect condition dataset 322 comprises one or more instances of the suspect condition data 305 which may have originated, for example, from evaluation of one or more instances of the re-certification trigger 332 and/or the application of an assessment ruleset 304. Operation 712 determines if an additional trigger condition (e.g., a second instance of the re-certification trigger 332) is met. If an additional instance of the re-certification trigger 332 is met, operation 712 returns to operation 708. If no additional instances of the re-certification trigger 332 are met, operation 712 proceeds to operation 714 which stores the suspect condition dataset 322. The suspect condition dataset 322 may be stored in a suspect condition database 320 and/or may be stored in a partition 240. The partition may be a separate database instance and/or may be a segregated record in the EMR 212 that may be clearly specified as undiagnosed. Operation 714 then proceeds to FIG. 9.

FIG. 8 is an assessment process flow 850 for determining a suspect condition from application of the assessment ruleset 304 to the health data 207 extracted from the EMR 212, according to one or more embodiments. Operation 800 extracts health data 207 from the EMR 212 of a patient 102 and stores the health data 207 in a computer memory (e.g., the computer memory 303). The health data 207 may include tests, records, documents, notes, and any other medical data stored in the EMR 212. The health data 207 may include data of one or more instances of a condition record 226 documenting a health condition of the patient 102. For example, where the condition record 226 stores data about a cancer the patient 102 was diagnosed with, the data extracted may include all physicians visited by the patient, one or more care plans, including all treatments undergone (including detailed information about prescriptions, dosages, time periods, side effects), tracking information for each stage of the cancer, and other similar health information. Referring to the embodiment of FIG. 2, the health data 207 extracted may include a condition name 228, a description 230, one or more instances of a condition ID 232, a qualitative test result 216 (which may include medical images such as CT scans and x-rays), a quantitative test result 218 (including but not limited to laboratory test results), a certification date 234, one or more instances of a document 236, and/or one or more audit logs 224.

Operation 802 applies an assessment ruleset 304 to the health data 207, for example by retrieving an instance of the assessment ruleset 304 (e.g., from a ruleset database, not shown in the figures), and then comparing one or more rules of the assessment ruleset 304. Operation 804 determines whether a rule of the assessment ruleset 304 generates an output and/or a positive result when applied to the health data 207. If the rule does not generate a result, operation 804 proceeds to operation 816. Where a result is generated from the assessment ruleset 304, operation 804 proceeds to operation 806. Operation 806 generates a suspect condition data 305, which may be assembled in the computer memory 303 from the health data 207 and/or other sources of data. For example, where the assessment ruleset 304 generates as an output a first condition ID 232.1 (e.g., that may be a diagnosis code) indicating that the patient 102 may have hypothyroidism, a second condition ID 232.2 (e.g., an HCC code) may be looked up and added to the suspect condition data 305.

Operation 808 determines whether the suspect condition already exists in the EMR 212, as may be specified in a condition record 226. For example, a comparison of and/or search for a diagnosis code or HCC code may be made. Where operation 808 determines the EMR 212 already includes a record diagnosing the suspect condition, and such suspect condition was generated as a result of a re-certification trigger 332, operation 808 proceeds to operation 810 which deletes the suspect condition data 305 and possibly logs the determination. Operation 810 then proceeds to operation 816. However, where option 808 determines no existing health condition in the EMR 212, operation 808 proceeds to operation 812.

Operation 812 determines whether the suspect condition is specified in the EMR 212 as not an existing condition in the patient 102. For example, operation 812 may compare a condition ID 232 to one or more audit logs 224 to determine a health condition was assessed but was certified to be absent. In another example, operation 812 may check for a piece of data that if present would preclude the health condition from existing in the patient 102 (e.g., data specifying the patient 102 is female for a health condition only affecting a male patient 102). In one or more embodiments, operation 812 may prevent a false positive. In just one example, a rule may exist that a patient 102 with diagnosed diabetes who is over a certain age and has a body-mass index exceeding a threshold value may have a suspect condition of hypertension. However, operation 812 may determine that the patient 102 recently had blood pressure measured and showed no signs of hypertension. Detection of a false positive results in operation 812 proceeding to operation 810. Otherwise, operation 812 proceeds to operation 814. Operation 814 adds the suspect condition data 305 and/or a suspect reasoning data 307 to a suspect condition dataset 322, similar to operation 710 of FIG. 7. Operation 816 determines if each applicable rule of the assessment ruleset 304 has been assessed. If not, operation 816 returns to operation 804 which assesses an additional rule. If all rules of the assessment ruleset 304 have been assessed, operation 816 proceeds to operation 818 which may operate similarly to operation 714 of FIG. 7. Operation 816 then may end or proceed to the process flow of FIG. 9.

FIG. 9 is a suspect condition data process flow 950 illustrating determination of a relevant encounter for presentation of the suspect condition data 305 and transmission of the suspect condition data 305, for example to the computing device 400 of FIG. 4, according to one or more embodiments. Operation 900 authenticates a clinician 104 or another healthcare professional qualified to diagnose the suspect condition. For example, the clinician 104 may be authenticated with a password, biometric, and/or physical access device (e.g., fob). A login event and/or authentication event may be logged in a log of the EMR management application 204. Operation 902 detects an EMR request 405 from a POC application 402. For example, the EMR request 405 may be passed through one or more systems on the way to the EMR management application 204, and/or the EMR management application 204 may send a communication over the network 101 to one or more other systems upon receiving the EMR request 405.

Operation 904 determines whether the EMR request 405 was made within the context of a native encounter 106 between the patient 102 and the clinician 104. For example, the authentication and/or login of the clinician 104 may specify a type of interaction or encounter between the clinician 104 and patient 102. In another example, the clinician 104 may be asked to verify (e.g., on the user interface 411), that he or she is in live communication and/or percipient with the patient. In yet another example, login or session information for the patient 102 and the clinician 104 may be logged to show the encounter is occurring over an audio or video feed. Where the encounter type is not a native encounter 106, operation 904 may terminate. Where the native encounter 106 is detected, operation 904 proceeds to operation 906.

Operation 906 generates an audit log 224 that the patient 102 and the clinician 104 are engaging in a native encounter 106. The audit log 224 may specify that the patient 102 arrived for their appointment and saw the clinician 104, engaged in a session with live text messages, an audio feed, and/or a video feed. Operation 908 reads the stored suspect condition dataset 322. The read operation may pull one or more suspect condition data 305 of the suspect condition dataset 322 into computer memory 303. Operation 910 then transmits the suspect condition dataset 322 and/or one or more suspect condition data 305 of the suspect condition dataset 322 to the computing device 400 of the clinician 104. In one or more embodiments, only relevant suspect condition dataset 322 may be extracted, as may be determined through reference between data of the suspect condition data 305 and the clinician 104 and/or other factors. For example, in in or more embodiments, only suspect condition data 305 that would be within the specialty of the clinician 104. Operation 910 ends, or proceeds to the process flow of FIG. 10.

FIG. 10 W is a certification process flow 1050 illustrating generation of certification options 313 for integration along with the suspect condition data 305 in the POC application 402 of FIG. 4, including a certification option, a diagnosis confirmation option, an absence confirmation option, a test order option, and a referral order option, according to one or more embodiments. Operation 1000 populates a user interface (e.g., the user interface 411) with health data (e.g., the health data 207) and/or data specifying one or more instances of a diagnosed condition (the diagnosed condition data 205). The populated data may be presented as part of the clinical documentation workflow 406. Operation 1002 generates certification options 313 to be proffered to the clinician 104 in association with the suspect condition. A standard set of certification options 313 may apply, or custom instances may be selected based on factors such as the suspect condition data 305, the clinician 104, the healthcare provider, etc. Operation 1004 populates a user interface (e.g., the user interface 411) with the suspect condition data 305 in visual association with the diagnosed condition data 205. For example, the suspect condition data 305 and the diagnosed condition data 205 may be populated in the same or adjacent feeds, windows and/or other visual elements.

Operation 1006 through operation 1015 illustrate example certification options. Although Operation 1006 through operation 1015 are shown in a sequence, the clinician 104 may select zero or more of the certification options 313. For example, selection of the certification of absence in operation 1012 does not require that the clinician 104 affirmatively select any other certification option 313, according to one or more embodiments.

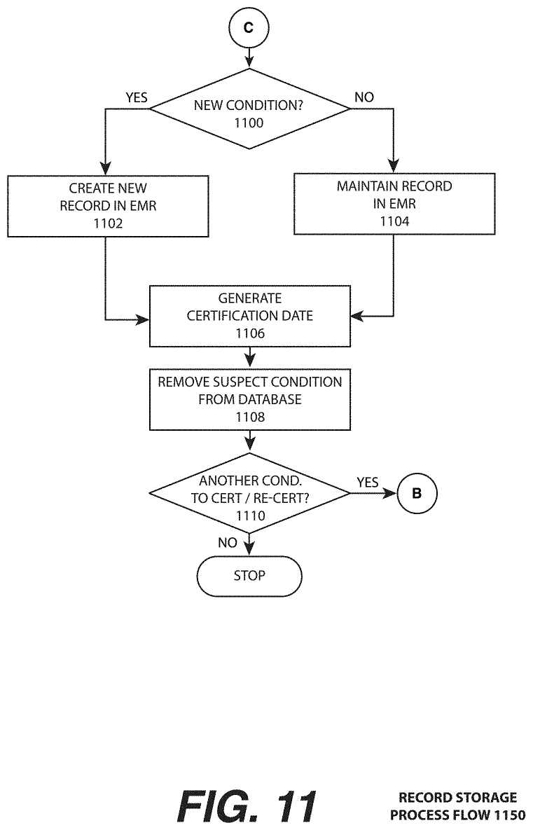

Operation 1006 determines whether a selection for condition certification has occurred. For example, the clinician 104 may select the certification option if the clinician 104 can easily confirm that the patient 102 has the health condition (e.g., from a distinct rash and/or other condition signature) and/or may select the certification option if the clinician 104 can tell the patient has the health condition after reviewing the data of the EMR 212, including the one or more diagnosed conditions shown in operation 1000. Upon selection of the certification option, operation 1006 may proceed to the process flow of the embodiment of FIG. 11. Operation 1008 determines whether additional information is to be collected. If selected, operation 1008 proceeds to operation 1009 where updated health data (e.g., the health data 207B) may be collected from the patient 102. For example, the clinician 104 may electronically enter a number of notes, may be prompted for open-ended questions (without being a formal or recognized test for diagnosis), and/or receive data from a patient 102 controlled data source. The updated health data 207B may then be optionally re-assessed. The additional information may or may not be entered in the EMR 212. Operation 1009 then may proceed to operation 802 of FIG. 8.

Operation 1010 determines whether the clinician 104 made a selection to order a test. The clinician 104 may be able to enter a test in free-form text, or may be able to select a test, e.g., from a drop-down menu or window. In one or more embodiments, a test database 338 may be referenced to determine a relevant test matching the suspect condition (e.g., possibly able to contribute toward a diagnosis of the suspect condition). The test may yield a quantitative test result 216 and/or a qualitative test result 218. Operation 1011 determines whether the test can be collected during the native encounter 106, for example a simple blood glucose level test or a psychiatric questionnaire and/or evaluation. If the test is collected within the native encounter 106 and the data is available, operation 1011 proceeds to operation 1009 where the test is undertaken. Otherwise, operation 1011 proceeds to operation 1016.

Operation 1012 processes a selection that certifies absence of a suspect condition. Where the absence certification is selected, operation 1012 proceeds to operation 1013, which may log and/or discard the suspect condition data 305 of the suspect condition. The audit log 224 may be stored in the EMR 212 of the patient 102 or may be stored in a audit database 250. Operation 1013 then may proceed to operation 1016.

Operation 1014 determines whether a referral option has been selected. Operation 1015 then queries a database containing a directory for a referral healthcare provider (e.g., the clinician database 336 of FIG. 2). Operation 1015 may present one or more options to the clinician 104, and upon receiving a selection and referring the patient 102, proceed to operation 1016. Operation 1016 determines whether another suspect condition is available to certify and/or recertify. If another suspect condition data 305 is available and/or requires a certification option, operation 1016 returns to operation 1000. Otherwise, operation 1016 ends.

Although not shown in the embodiment of FIG. 10, a second selection of the certification options 313 may be made. For example, after operation 1015 but before operation 1016, an operation may be a decision that determines whether an additional selection may be made, in which case such operation may return to operation 1004. In one or more other embodiments, if no selection is made (e.g., because the clinician 104 forgot or the POC application 402 was shut down before a selection could be made), the suspect condition data 305 may persist in the suspect condition dataset 322 until it may be presented at a next instance of the native encounter 106 and/or reviewed in another way.

FIG. 11 is a record storage process flow 1150 illustrating a process by which a suspect condition that was converted to a diagnosed condition may be stored as a new record (e.g., a new instance of the condition record 226) and/or a certification date 234 of an existing record (e.g., a existing instance of the condition record 226) in the EMR 212, according to one or more embodiments. Operation 1100 determines whether the suspect condition of the suspect condition data 305 is a new condition or an existing condition. For example, an existing condition may be a health condition triggered by the re-certification trigger 332 and/or generated by the process of FIG. 7. A new suspect condition may have been output from application of the assessment ruleset 304 to the health data 207 and/or by the process of FIG. 8. Where the suspect condition is a new health condition, operation 1100 proceeds to operation 1102 which creates a new record in the EMR 212 (e.g., a new instance of the condition record 226). Optionally, one or more documentation requirements (e.g., the documentation requirement 509) may be presented to the clinician 104 to assist in documenting the diagnosis. Where the suspect condition is an existing health condition, operation 1100 proceeds to operation 1104 representing maintenance of the existing record in the EMR 212. Optionally, one or more documentation requirements (e.g., the documentation requirement 509) may be presented to the clinician 104 to assist in documenting the re-certification of the diagnosis, including but not limited to any new documentation requirements 509 that may be triggered by the re-certification trigger 332. Operation 1102 and operation 1104 then proceed to operation 1106 which updates and/or adds a certification date (e.g., the certification date 234) of the EMR 212. Where the suspect condition has an existing condition record 226 in the EMR 212 with an existing instance of the certification date 234.1, operation 1106 may add an updated instance of the certification date 234.2. Operation 1108 removes the suspect condition data 305 from the suspect condition dataset 322 and/or the partition 240, which may reduce the suspect condition dataset 322 to include only suspect conditions that have yet to be evaluated. Operation 1110 determines whether additional suspect conditions have yet to be certified, as may be determined by existing suspect condition data 305 within the suspect condition dataset 322. Where no suspect condition data 305 remains, operation 1110 ends. Otherwise, operation 1110 may return to operation 1000 of FIG. 10.

FIG. 12 is a documentation assessment process flow 1250 illustrating an automatic evaluation of documentation of the diagnosed condition (e.g., the diagnosed condition resulting from certification and/or re-certification of the suspect condition), for example to ensure insurance documentation requirements, according to one or more embodiments. Operation 1200 determines a documentation requirement 509. The documentation requirement 509 may be specified in the documentation requirement database 506, as shown and described in conjunction with the embodiment of FIG. 5. The documentation requirement 509 may be data that specifies any documentation requirement of the health condition, for example an internal quality control, a best practice based on a national standard, an insurance reimbursement requirement, a government regulation, etc. The documentation requirement 509 may be associated with a condition through a condition ID 232. The documentation requirement 509 may specify one or more standard tests, results, completed forms, or other diagnosis requirement. Operation 1202 queries the EMR 212 for a document 236 matching the documentation requirement 509 of the diagnosed condition. For example, the document 236 may be the qualitative test result 218, the quantitative test result 216, at least one note made by a clinician 104, a specified care plan, a record of a prescription medication prescribed at the time of diagnosis, etc. Comparison of document identifiers may be used (including standard form numbers or identifiers), examination of electronic file metadata, and/or voice, text, or image analysis. For example, an image analysis process may be conducted to ensure a CT scan was conducted, or an MRI with a specific type of contrast dye.

Operation 1204 determines a documentation deficiency of the diagnosed condition based on a failure to return the document 236 matching the documentation requirement 509. For example, an error may be returned from the EMR management application 204 that specifies that no data matched the query of the EMR database 210 generated by operation 1202. In another example, one or more instances of the document 236 may be returned but may be determined through additional processes not to match the documentation requirement 509. For example, a medical image may be returned but analyzed and determined to meet a resolution requirement for a diagnosis. In another example, the document 236 may be returned and data of the document 236 may be transmitted to a third party to determine if the third party has an associated set of data required to properly and/or fully meet the documentation requirement 509.

Operation 1206 may generate a deficiency notification 505. The deficiency notification 505 is data that may include an identifier of the required documentation, an explanation of why the documentation is required, and/or a reference to one or more procedures for obtaining the required documentation. Operation 1208 detects an EMR request 405 generated by a POC application 402 running on a computing device 400 of a clinician 104 requesting information of the EMR 212 of the patient 102, where the POC application 402 comprises a clinical documentation workflow 406. Operation 1208 may have occurred previous to generation of the deficiency notification 505, for example prior to the conversion of the suspect condition to the diagnosed condition with the assessed documentation requirement 509 in operation 1200. Operation 1210 integrates the deficiency notification 505 in visual association with the diagnosed condition within the native encounter 106 to direct the clinician 104 to obtain the document 236 meeting the documentation requirement 509 before the native encounter 106 of the clinician 104 and the patient 102 ends.

FIG. 13 illustrates a first part of an example embodiment in which a clinician 104 re-certifies a diabetes diagnosis and certifies diabetes-related complications. In the example embodiment of FIG. 13, a patient 102 (“John”) may engage in a native encounter 106 with a clinician 104 (“Sarah”) such as an in-person appointment at a primary care clinic. The clinic operates within a healthcare provider.

After a short wait for John in a waiting room, Sarah enters and greets John. It is a busy day at Sarah's practice, and John and Sarah will only have about 20 minutes together. Sarah logs in to a point-of-care application running on her iPad (e.g., the POC application 402 running on the computing device 400). She retrieves health information (e.g., the health information 239) of John's electronic medical record 212 through the POC application 402. In the retrieval process an audit log 224 is automatically generated specifying that Sarah is meeting with John in person in her office.

Prior to Sarah requesting John's electronic medical record 212, a set of health data 207 extracted from John's EMR 212 is analyzed for either (i) conditions that he either was diagnosed with in the past but that now need to be re-certified, to check if he continues to have such condition, and/or (ii) any new conditions which John may not have yet been diagnosed with. In this example, a condition re-certification routine 308 running on a server determined that John's diabetes has not received a diagnosis re-certification in over a year, activating a re-certification trigger. Diabetes is therefore a suspect condition electronically stored as a suspect condition data 305.1. In addition, a suspect condition assessment engine 306 applied an assessment ruleset 304 to John's health data 207. Because John has diabetes, the assessment ruleset 304 includes a number of rules that may identify certain events or other conditions that could indicate diabetes-related complications. In this example, the assessment ruleset 304 include a rule that if a patient 102 has diabetes and two or more records (e.g., physician notes) in the past three months include the term “fall”, “trip,” or a similar term, then the patient 102 may suffer from diabetes-related nerve damage in their feet. John had two such incidents recently, one in which a bad fall lead to a shin laceration. Therefore, a suspect condition data 305.2 was generated and stored.

Sarah's iPad generates an EMR request 405 to retrieve health information 239 from John's EMR 212. For example, the health information 239 may include all or some of the condition records 226 in John's EMR 212, including the most important records (e.g., diabetes) and the most recent records (e.g., a shin laceration). About the same time that the EMR request 405 is received by the record server 200, such request is detected by a certification server 300. The certification server 300 may then read both the suspect condition data 305.1 and the suspect condition data 305.2 from a database and send it with the health information 239.

An initial user interface 411 is shown in FIG. 13. The health information 239.1 sets out John's major health condition and provides a link to detailed records that can also be retrieved from the EMR 212, (e.g., should Sarah need to look deeper into his medical history. However, immediately below the health information 239.1 in the user interface 411 is displayed the suspect condition data 305.1. Sarah is given two alerts. First, that John's diabetes was last diagnosed over a year ago and that there is an annual re-certification requirement. This re-certification requirement is important to John so his conditions adequately cared for. The re-certification requirement also may be important to the healthcare provider so it is adequately reimbursed for John's care. Second, a documentation requirement 509 may require that any certification of diabetes now requires a new glycated hemoglobin (“A1C”) test. As such, a deficiency notification 505 may be generated and also presented. Several certification options 313 are generated for the suspect condition data 305.1, but include a certification option, a testing option, and a referral option. In this example, the certification option is not initially selectable (e.g., “greyed-out”) because the new A1C test is required. Sarah may not have known about the new A1C testing requirement before seeing the alert, nor have remembered the last time John received a condition certification for diabetes.

Therefore, Sarah selects the “test” button and administers the A1C test. After entry of the results, which are recorded in the EMR 212, Sarah is then able to select the “certify” option. The EMR 212 may update the condition record 226 for John's diabetes with a new certification data 234 specifying the date Sarah re-certified John: February 20, 2021.

Sarah then scrolls down in the user interface 411 and notices a warning. A couple months before, John suffered a serious cut after he tripped while walking down a staircase. The health information 239.2 is presented in the user interface 411 along with relevant notes. A suspect condition data 305.2 is presented below the health information 239.2, specifying that it is a possibility that John has diabetic peripheral neuropathy, a diabetes-related complication resulting in nerve damage to the extremities. The health condition can make it difficult for ones' feet to feel the ground, and therefore increase the chance of tripping and/or falling. A suspect reasoning data 307.2 explains the rule of the assessment ruleset 304, in narrative form, that lead to the suspect condition of diabetic peripheral neuropathy.

Sarah examines the notes from John's emergency room visit. She does not see anything that would specifically indicate John was having trouble sensing his feet. Sarah therefore decides to select the “collect additional info” option of the set of certification options 313.2. A notepad appears in which Sarah can input notes while interviewing John. Sarah begins to ask John about the incidents, including whether he has recently felt any tingling in his feet.

In response, John explains “I remember tripping over the second or third stair step, but I′m not sure if it was because I could not feel the step. I might have had trouble seeing it, my eyes are not as good anymore.” Sarah takes corresponding notes through her iPad, which are entered in John's EMR 212.

Sarah next selects “test”, and a set of sub-options are automatically generated, including one test option that is a qualitative test recently approved by the American Diabetes Association and another older qualitative test using a filament to test nerve sense. The first qualitative test includes a questionnaire with a number of yes-or-no questions and asking the patient 102 to provide ratings on a subjective scale of one to five in response to various questions. First, Sarah administers the questionnaire test, and she inputs the results in her iPad. Second, Sarah follows the procedure to conduct the filament test by brushing a soft nylon fiber (monofilament) over areas of John's skin to test touch sensitivity.

Sarah concludes that John is indeed developing diabetic peripheral neuropathy. She selects the confirmation option (“certify”). A new condition record 226 is set up in John's EMR 212, including the qualitative test result 216 for both the questionnaire and the filament test, Sarah's physician notes, and possibly other relevant documents. Sarah explains it is good that the condition was identified when it was so that a care plan could be developed. John did not understand why he had fallen. Both Sarah and John are relieved the condition was identified so it can be addressed. However, before Sarah and John can end the appointment, Sarah receives another notification.

FIG. 14 illustrates a second part of the example of FIG. 13, according to one or more embodiments. In the process of diagnosing John with diabetic peripheral neuropathy, John made a statement that he was experiencing trouble with eyesight. In the present example, the healthcare provider may have a server running an automatic process to re-apply the assessment ruleset 304 as new information becomes available, especially in a period after a native encounter 106 of the patient was initiated (e.g., the realtime assessment agent 316). The new information may be referred to as the updated health data 207B (where the original health data is therefore referred to as the health data 207A). The assessment ruleset 304 may include a rule that if a patient 102 has diabetes and if any records after the diabetes diagnosis implicates eyesight impairment, then the patient 102 may have diabetes-related diabetic retinopathy.

The suspect condition 305.3 is generated, along with the reasoning data 307.3. As a general practitioner, Sarah is likely not to have a fundus camera that may be generally utilized to diagnose diabetic retinopathy and/or the qualifications or experience to do so. In generating the certification options 313, the server may identify Sarah's general practice, and therefore present a referral option. Sarah selects the “refer” option and a list of qualified ophthalmologists within a network of the healthcare provider are presented. She lets John know it is important for him to set up an appointment right away for an eye exam and completes a referral workflow. John and Sarah end the appointment having identified at least one and possibly another important health condition.

In the present example, one or more elements of the certification network 100 assisted Sarah in accurately and/or efficiently diagnosing John. Sarah was also easily reminded to re-certify John's condition. Sarah was able to easily navigate suspect conditions and assess them relative to existing health information from the EMR 212 of the patient 102. Sarah could access tests relevant to diagnosing John's suspected condition of diabetic peripheral neuropathy while he was present in a native encounter 106. Sarah was able to have a suspect condition brought to her addition that she did not regularly diagnose (i.e., diabetic retinopathy), and easily refer the suspect condition to a colleague who could complete a diagnosis critical to John's health. Sarah was easily made aware of a new change in approved diagnosis procedure (A1C) adopted by the healthcare provider, as it could otherwise be difficult for a healthcare provider to communicate new standards it adopts to its healthcare professionals.

At the organizational level, there may be increased efficiency of healthcare professionals such as Sarah, helping their appointments stay on time while still addressing all relevant health conditions (including some not previously identified). There may be a decreased risk of malpractice liability because a doctor within the emergency room had not previously recognized John's fall could stem from a diabetes-related complication. Without diagnosing John's condition, John may not have been aware of the seriousness of his diabetes progression, possibly causing greater harm to John without immediately beginning remediation steps. The healthcare provider may also receive full insurance reimbursement claims for the quality healthcare provided to John, for example because the insurance company offers a quality or risk bonus to providers treating high-risk patients like John. As a result, the healthcare provider may have an increase in patient satisfaction, brand goodwill, and revenue.

In the embodiment of FIG. 13 and FIG. 14, there may be additional aspects of the present embodiments. For example, in assessing diabetic peripheral neuropathy, Sarah may select “collect additional info” which may provide an option for downloading data from a health tracking device of the patient 102 to generate the updated health data 207B. The health tracking device may be, for example, a smartwatch (e.g., Apple® Watch), a fitness tracking device (e.g., a FitBit®, a Bellabeat Leaf®, etc.) and/or a health tracking service that may store health information outside the EMR 212 under control of the patient. The downloaded data may be directly transmitted to the computing device 400 (e.g., via Bluetooth®), which may then be related to the record server 200, or may find its way to the record server 200 through other means such as the patient 102 authorizing transmission through an API. In the example of FIG. 13, John's daily movement data of the health tracking device from an accelerometer could be assessed to indicate stumbles, trips, and/or falls. Analysis of the data could increase the likelihood the patient has diabetic peripheral neuropathy, and therefore secure a more efficient and/or accurate diagnosis or more robust documentation.

Although the present embodiments have been described with reference to specific example embodiments, it will be evident that various modifications and changes may be made to these embodiments without departing from the broader spirit and scope of the various embodiments. For example, the various devices, engines, agent, routines, and modules described herein may be enabled and operated using hardware circuitry (e.g., CMOS based logic circuitry), firmware, software, or any combination of hardware, firmware, and software (e.g., embodied in a non-transitory machine-readable medium). For example, the various electrical structure and methods may be embodied using transistors, logic gates, and electrical circuits (e.g., application specific integrated circuitry (ASIC) and/or Digital Signal Processor (DSP) circuitry).

In addition, it will be appreciated that the various operations, processes, and methods disclosed herein may be embodied in a non-transitory machine-readable medium and/or a machine-accessible medium compatible with a data processing system (e.g., the record server 200, the certification server 300, the computing device 400, the documentation server 500). Accordingly, the specification and drawings are to be regarded in an illustrative rather than a restrictive sense.

The structures in the figures such as the engines, routines, and modules may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the preceding disclosure.

Embodiments of the invention are discussed above with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures.

From reading the present disclosure, other variations and modifications will be apparent to persons skilled in the art. Such variations and modifications may involve equivalent and other features which are already known in the art, and which may be used instead of or in addition to features already described herein.

Although claims have been formulated in this application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any claim and whether or not it mitigates any or all of the same technical problems.

Features which are described in the context of separate embodiments may also be provided in combination in a single embodiment. Conversely, various features which are, for brevity, described in the context of a single embodiment, may also be provided separately or in any suitable sub-combination. The applicants hereby give notice that new claims may be formulated to such features and/or combinations of such features during the prosecution of the present application or of any further application derived therefrom.

References to “one embodiment,” “an embodiment,” “example embodiment,” “various embodiments,” “one or more embodiments,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least one or more embodiments of the invention” includes the stated particular feature, structure, or characteristic.

The enumerated listing of items does not imply that any or all of the items are mutually exclusive, unless expressly specified otherwise.

It is understood that the use of a specific component, device and/or parameter names are for example only and not meant to imply any limitations on the invention. The invention may thus be implemented with different nomenclature and/or terminology utilized to describe the mechanisms, units, structures, components, devices, parameters and/or elements herein, without limitation. Each term utilized herein is to be given its broadest interpretation given the context in which that term is utilized.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; a smartphone, application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, one or more embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java.TM., Jini.TM., C, C++, Smalltalk, Perl, UNIX Shell, Visual Basic or Visual Basic Script, Virtual Reality Markup Language (VRML), ColdFusion.TM. or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

The term “processor” may refer to any device or portion of a device that processes electronic data from registers and/or memory to transform that electronic data into other electronic data that may be stored in registers and/or memory. A “computing platform” may comprise one or more processors.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Having fully described at least one embodiment of the present invention, other equivalent or alternative methods of implementing the certification network 100 according to the present invention will be apparent to those skilled in the art. Various aspects of the invention have been described above by way of illustration, and the specific embodiments disclosed are not intended to limit the invention to the particular forms disclosed. The particular implementation of the loyalty rewards programs may vary depending upon the particular context or application. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

We claim:

1. A method for accurately and efficiently confirming a suspected health condition of a patient, comprising:
extracting a health data of the patient from an electronic medical record of the patient, the health data selected from any one of a diagnosed condition, a quantitative test result, and a qualitative test result;
applying an assessment ruleset to the health data of the patient that outputs a suspect condition based on the health data, where the suspect condition is a potential health condition diagnosable by a clinician;
generating a suspect condition data comprising a name of the suspect condition, a description of the suspect condition, and a hierarchical condition category code;
detecting an EMR request generated by a documentation application running on a computing device of a clinician requesting information of the electronic medical record of the patient, where the documentation application comprises a clinical documentation workflow;
transmitting information of the electronic medical record along with the suspect condition data from a server to the computing device over a network, where the information of the electronic medical record includes one or more diagnosed conditions and is presented to the clinician through a user interface of the POC application within the clinical documentation workflow;
integrating the suspect condition data in visual association with the one or more diagnosed conditions extracted from the electronic medical record within the clinical documentation workflow to provide a context for the suspect condition relative to the one or more diagnosed conditions within a native encounter of the clinician and the patient;
receiving a diagnosis confirmation of the suspect condition from the computing device of the clinician to certify the suspect condition; and
converting the suspect condition into the diagnosed condition based on the context for the suspect condition relative to the one or more diagnosed conditions within the native encounter by generating a new record in the electronic medical record of the patient.

2. The method of claim 1, further comprising:
generating a suspect reasoning data specifying one or more reasons the suspect condition was output from application of the assessment ruleset to the health data of the patient; and
integrating at least one of the suspect reasoning data and a reference to the suspect reasoning data in visual association with the one or more diagnosed conditions extracted from the electronic medical record within the clinical documentation workflow.

3. The method of claim 2, further comprising:
determining that the suspect condition is a care gap of the patient through comparison to at least one of the health data and the one or more diagnosed conditions in the electronic medical record.

4. The method of claim 3, further comprising:
generating a set of certification options in association with the suspect condition data on the user interface, the set of certification options comprising a diagnosis confirmation option and at least one of:
(i) an absence confirmation option initiating generation of an absence record certifying that the suspect condition is absent from the patient,
(ii) a referral order option initiating a referral process to refer the patient to a referral clinician qualified to diagnose the suspect condition,
(iii) a test order option to initiate ordering a diagnostic test approved to diagnose the suspect condition, and
(iv) an information collection option to initiate a data collection process gathering additional information usable to diagnose the suspect condition within the native encounter of the clinician and the patient;
referencing a clinician database to determine the referral clinician qualified to diagnose the suspect condition;
referencing a test database to determine the diagnostic test approved to diagnose the suspect condition; and
referencing a diagnostic procedure database to extract a data collection process usable to diagnose the suspect condition.

5. The method of claim 4, further comprising:
initiating at least one of the data collection process gathering additional information usable to diagnose the suspect condition and the diagnostic test approved to diagnose the suspect condition;
collecting an updated health data from the patient comprising at least one of the data generated from the additional information usable to diagnose the suspect condition and a test result of the diagnostic test;
re-applying the assessment ruleset to the updated health data of the patient to output a second suspect condition based on the updated health data; and
integrating a second suspect condition data in visual association with the suspect condition within the clinical documentation workflow to provide the context for the suspect condition relative to the second suspect condition within the native encounter of the clinician and the patient.

6. The method of claim 5, further comprising:
determining a documentation requirement of at least one of the suspect condition and the diagnosed condition;
querying the electronic medical record for a document matching the documentation requirement of the diagnosed condition;
determining a documentation deficiency in the diagnosed condition based on a failure to return the document matching the documentation requirement;
generating a deficiency notification; and
optionally integrating the deficiency notification in visual association with the diagnosed condition within the native encounter to direct the clinician to obtain the document meeting the documentation requirement before the native encounter of the clinician and the patient ends.

7. The method of claim 6, further comprising:
generating an audit log that the patient is in the presence of the clinician;
storing the suspect condition data in a partition of the electronic medical record distinct from the one or more diagnosed conditions; and
receiving data of a health tracking device of the patient to generate the updated health data of the patient,
wherein the suspect condition data further comprising a condition ID that is at least one of a hierarchical condition code and a diagnosis code,
wherein the patient UID is at least one of a medical record number, a clinical record number, and a record set ID,
wherein the quantitative test result comprises data from a physical characteristic of the patient, a vital sign, and a laboratory test,
wherein the quantitative test result is received in a communication protocol comprising at least one of FHIR and HL7,
wherein the qualitative test result comprises data from family history evaluation, physical exam, a patient generated report, a system review, a verbal screening, a written screening, an observational narrative, a psychiatric evaluation, a medical imaging data, and a medical graph data,
wherein the documentation requirement comprises the test result, a clinician narrative, an identification of multiple related health conditions, a certification date, a clinician authentication, a care plan, and
wherein the documentation requirement is based on data specifying at least one of an internal quality control, a new diagnosis procedure, an insurance claim requirement, and a government regulation.

8. A method for accurately and efficiently confirming a suspected health condition of a patient, comprising:
extracting a health data of the patient from an electronic medical record of the patient, the health data comprising a diagnosed condition;
determining occurrence of a re-certification trigger, the re-certification trigger comprising a certification date of the diagnosed condition exceeding a threshold time to define a suspect condition, where the suspect condition is a potential health condition that may be continuing in the patient and is diagnosable by a clinician;
generating a suspect condition data comprising a name of the suspect condition, a description of the suspect condition, and a hierarchical condition category code;
detecting an EMR request generated by a documentation application running on a computing device of a clinician requesting information of the electronic medical record of the patient, where the documentation application comprises a clinical documentation workflow;
transmitting information of the electronic medical record along with the suspect condition data from a server to the computing device over a network, where the information of the electronic medical record includes one or more diagnosed conditions and is presented to the clinician through a user interface of the POC application within the clinical documentation workflow;
generating an audit log that the patient is in the presence of the clinician;
integrating the suspect condition data in visual association with the one or more diagnosed conditions extracted from the electronic medical record within the clinical documentation workflow to provide a context for the suspect condition relative to the one or more diagnosed conditions within a native encounter of the clinician and the patient;
receiving a diagnosis confirmation of the suspect condition from the computing device of the clinician to re-certify the diagnosed condition; and
conserving the diagnosed condition by maintaining an existing instance of a condition record of the diagnosed condition in the electronic medical record of the patient while adding a new certification date associated with the existing instance of the condition record.

9. The method of claim 8, further comprising:
determining a documentation requirement of the diagnosed condition;
querying the electronic medical record for a document matching the documentation requirement of the diagnosed condition;
determining a documentation deficiency in the diagnosed condition based on a failure to return the document matching the documentation requirement;
generating a deficiency notification; and
optionally integrating the deficiency notification in visual association with the diagnosed condition within the native encounter to direct the clinician to obtain the document meeting the documentation requirement before the native encounter of the clinician and the patient ends.

10. The method of claim 9, further comprising:
generating a re-certification explanation specifying one or more reasons the suspect condition was subject to the re-certification trigger; and
integrating at least one of the re-certification explanation and a reference to the re-certification explanation in visual association with the one or more diagnosed conditions extracted from the electronic medical record within the clinical documentation workflow.

11. The method of claim 10, further comprising:
wherein the re-certification trigger further comprising data specifying at least one of a change of a care provider of the patient, and a change in a care program requiring a new quality measure.

12. The method of claim 11, further comprising:
generating a set of certification options in association with the suspect condition data on the user interface, the set of certification options comprising a diagnosis confirmation option and at least one of:
(i) an absence confirmation option initiating generation of an absence record certifying that the suspect condition is absent from the patient,
(ii) a referral order option initiating a referral process to refer the patient to a referral clinician qualified to diagnose the suspect condition,
(iii) a test order option to initiate ordering a diagnostic test approved to diagnose the suspect condition, and
(iv) an information collection option to initiate a data collection process gathering additional information usable to diagnose the suspect condition within the native encounter of the clinician and the patient;
referencing a clinician database to determine the referral clinician qualified to diagnose the suspect condition;
referencing a test database to determine the diagnostic test approved to diagnose the suspect condition; and
referencing a diagnostic procedure database to extract a data collection process usable to diagnose the suspect condition.

13. The method of claim 12,
wherein the re-certification trigger further comprising data specifying at least one of a change of a care policy of the patient, and a change in a healthcare regulation,
wherein the suspect condition data further comprising a condition ID that is at least one of a hierarchical condition code and a diagnosis code,
wherein the patient UID is at least one of a medical record number, a clinical record number, and a record set ID,
wherein the quantitative test result comprises data from a physical characteristic of the patient, a vital sign, and a laboratory test,
wherein the quantitative test result is received in a communication protocol comprising at least one of FHIR and HL7,
wherein the qualitative test result comprises data from family history evaluation, physical exam, a patient generated report, a system review, a verbal screening, a written screening, an observational narrative, a psychiatric evaluation, a medical imaging data, and a medical graph data,
wherein the documentation requirement comprises test result, a clinician narrative, an identification of multiple related health conditions, the certification date, a clinician authentication, a care plan, and
wherein the documentation requirement is based on data specifying at least one of an internal quality control, a new diagnosis procedure, an insurance claim requirement, a government regulation.

14. A system for accurately and efficiently confirming a suspected health condition of a patient, comprising:
an record server:
a processor of the record server,
a memory of the record server,
an EMR database storing an electronic medical record comprising a patient UID that is a unique identifier associated with the patient, a condition record, and a certification date of the condition record,
EMR management application for extracting information of the electronic medical record in response to a query and at least one of generating and modifying the condition record in response to a record creation instruction,
certification server, comprising:
a processor of the certification server,
a memory of the certification server,
a suspect condition assessment engine comprising computer readable instructions that when executed on the processor of the certification server:
request a health data of the patient from the electronic medical record of the patient, the health data selected from any one of a diagnosed condition, a quantitative test result, and a qualitative test result;
apply an assessment ruleset to the health data of the patient that outputs a suspect condition based on the health data, where the suspect condition is a potential health condition diagnosable by a clinician; and
generate a suspect condition data comprising a name of the suspect condition, a description of the suspect condition, and a hierarchical condition category code,
a request agent comprising computer readable instructions that when executed on the processor of the certification server:
detects an EMR request generated by a documentation application running on a computing device of a clinician requesting information of the electronic medical record of the patient,
a clinician notification routine comprising computer readable instructions that when executed on the processor of the certification server:
receives a call from the request agent, and
transmit the suspect condition data in coordination with information of the electronic medical record to the computing device over the network, where the information of the electronic medical record includes one or more diagnosed conditions and is presented to the clinician through a user interface of the POC application within a clinical documentation workflow, and
a network communicatively coupling the record server and the certification server.

15. The system of claim 14, further comprising:
the computing device of the clinician, comprising:
a processor of the computing device,
a memory of the computing device,
a display,
a request module comprising computer readable instructions that when executed on the processor of the computing device:
generate the EMR request, and
transmit the EMR request to the record server,
a diagnosis application comprising a clinical documentation workflow and the user interface,
an integration routine comprising computer readable instructions that when executed on the processor of the computing device:
integrate the suspect condition data in visual association with the one or more diagnosed conditions extracted from the electronic medical record within the clinical documentation workflow to provide a context for the suspect condition relative to the one or more diagnosed conditions within a native encounter of the clinician and the patient, and
a certification routine comprising computer readable instructions that when executed on the processor of the computing device:
generate a diagnosis confirmation of the suspect condition to at least one of certify and re-certify the diagnosed condition, and
generate an instance of the record creation instruction for creation of a new record in the electronic medical record of the patient upon certification of the diagnosed condition.

16. The system of claim 15, further comprising:
a documentation server, comprising:
a processor of the documentation server,
a memory of the documentation server,
a documentation requirement database comprising a documentation requirement of the diagnosed condition,
a documentation assessment engine comprising computer readable instructions that when executed on the processor of the documentation server:
query the documentation requirement database,
determine the documentation requirement of the diagnosed condition,
query the electronic medical record for a document matching the documentation requirement of the diagnosed condition,
determine a documentation deficiency in the diagnosed condition based on a failure to return the document matching the documentation requirement,
generate a deficiency notification, and
transmit the deficiency notification over the network.

17. The system of claim 16,
wherein the certification server further comprising:
a realtime assessment agent comprising computer readable instructions that when executed on the processor of the certification server:
detect an updated health data, and
calls the suspect condition assessment engine for application of the assessment ruleset to the updated health data and optionally the health data,
a re-certification condition routine comprising computer readable instructions that when executed on the processor of the certification server:
determine occurrence of a re-certification trigger,
generate a re-certification explanation specifying one or more reasons the suspect condition was subject to the re-certification trigger, and
generate a suspect condition data comprising a name of the suspect condition, a description of the suspect condition, and the hierarchical condition category code,
transmit the suspect condition data in coordination with information of the electronic medical record from a server to the computing device over a network, where the information of the electronic medical record includes one or more diagnosed conditions and is presented to the clinician through the user interface of the POC application within the clinical documentation workflow,
a certification date module comprising computer readable instructions that when executed on the processor of the certification server:
add a new certification date associated with the existing instance of the condition record, and
wherein the suspect condition assessment engine further comprising computer readable instructions that when executed on the processor of the certification server:
determine that the suspect condition is a care gap of the patient through comparison to at least one of the health data and the one or more diagnosed conditions in the electronic medical record, and
generate a suspect reasoning data specifying one or more reasons the suspect condition was output from application of the assessment ruleset to the health data of the patient.

18. The system of claim 17,
wherein the record server further comprising:
an authentication module comprising computer readable instructions that when executed on the processor of the record server:
authenticate at least one of the clinician and the computing device of the clinician,
an audit log routine comprising computer readable instructions that when executed on the processor of the record server:
generate an audit log that the patient is in the presence of the clinician, and
a partitioned storage routine comprising computer readable instructions that when executed on the processor of the record server:
generate a request for the EMR management application to store the suspect condition data in a partition of the electronic medical record distinct from the one or more diagnosed conditions.

19. The system of claim 18,
wherein the computing device further comprising:
an option population routine comprising computer readable instructions that when executed on the processor of the computing device:
generate a set of certification options in association with the suspect condition data on the user interface, the set of certification options comprising a diagnosis confirmation option and at least one of:
(i) an absence confirmation option initiating generation of an absence record certifying that the suspect condition is absent from the patient;
(ii) a referral order option initiating a referral process to refer the patient to a referral clinician qualified to diagnose the suspect condition;
(iii) a test order option to initiate ordering a diagnostic test approved to diagnose the suspect condition; and
(iv) an information collection option to initiate a data collection process gathering additional information usable to diagnose the suspect condition within the native encounter of the clinician and the patient; and,
wherein the integration routine further comprising computer readable instructions that when executed on the processor of the computing device:
integrate at least one of the suspect reasoning data and a reference to the suspect reasoning data in visual association with the one or more diagnosed conditions extracted from the electronic medical record within the clinical documentation workflow.

20. The system of claim 19,
wherein the documentation server further comprising:
an adjustment generation engine comprising computer readable instructions that when executed on the processor of the documentation server:
generates a reimbursement claim based on the diagnosed condition associated with the diagnosis confirmation, and
wherein the re-certification trigger comprising at least one of the certification date of the diagnosed condition exceeding a threshold time to define a suspect condition, data specifying at least one of a change of a care provider of the patient, a change in a care program requiring a new quality measure, a change of a care policy of the patient, and a change in a healthcare regulation,
wherein the suspect condition data further comprising a condition ID that is at least one of a hierarchical condition code and a diagnosis code,
wherein the patient UID is at least one of a medical record number, a clinical record number, and a record set ID,
wherein the quantitative test result comprises data from a physical characteristic of the patient, a vital sign, and a laboratory test,
wherein the quantitative test result is received in a communication protocol comprising at least one of FHIR and HL7,
wherein the qualitative test result comprises data from family history evaluation, physical exam, a patient generated report, a system review, a verbal screening, a written screening, an observational narrative, a psychiatric evaluation, a medical imaging data, and a medical graph data,
wherein the documentation requirement comprises test result, a clinician narrative, an identification of multiple related health conditions, the certification date, a clinician authentication, a care plan, and
wherein the documentation requirement is based on data specifying at least one of an internal quality control, a new diagnosis procedure, an insurance claim requirement, a government regulation.
